(19) United States
(12) Patent Application Publication
(10) Pub. No.: US 2003/0158213 A1
(43) Pub. Date: Aug. 21, 2003

(54) Xanthine oxidase inhibition as a strategy to alleviate oxidative impairment of vascular function

(76) Inventors: Bruce A. Freeman, Birmingham, AL (US); Margaret Tarpey, Birmingham, AL (US); Tom Ryan, Birmingham, AL (US); Tim Townes, Birmingham, AL (US); Mutay Aslan, Antalya (TR)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/298,739

(22) Filed: Nov. 18, 2002

Related U.S. Application Data
(60) Provisional application No. 60/333,268, filed on Nov. 16, 2001.

(51) Int. Cl. A61K 31/519

(52) U.S. Cl. 514/262.1

(57) ABSTRACT

Disclosed is a method for alleviating the oxidative impairment of vascular function by inhibiting the activity of xanthine oxidase, or active forms thereof. Xanthine oxidase levels have been shown to be increased by a variety of conditions, including sickle cell disease. In the present disclosure, allopurinol is used to inhibit xanthine oxidase activity. As a result of the inhibition of xanthine oxidase, .NO levels in a subject can be maintained. In addition to sickle cell disease, allopurinol inhibition of xanthine oxidase may be used to treat other conditions, including, but not limited to, respiratory distress, kidney disease, liver disease, ischemia-reperfusion injury, organ transplant, sepsis, burns, viral infections and hemorrhagic shock.

### FIELD OF THE DISCLOSURE

This disclosure claims the benefit of U.S. Provisional Patent Application No. 60/333,268, filed on Nov. 16, 2001. The present disclosure is directed to a method of using compounds which inhibit the activity of xanthine oxidase in order to alleviate the inhibition of vascular function caused by oxidative events and/or inflammatory conditions.

### BACKGROUND OF THE DISCLOSURE

The production of oxygen radical species, such as O₂. and H₂O₂, have been know to cause tissue injury in living organisms and contribute to a wide variety of disease processes. Multiple features of sickle cell disease (SCD) reveal that inflammatory-derived oxidative reactions lead to impaired nitric oxide (.NO)-dependent vascular function. Nitric oxide is a free radical mediator of neurotransmitter, cell-mediated immunity and tissue redox reactions. In regulating endothelial-dependent vascular relaxation, .NO diffuses to target cells to stimulate cGMP production by guanylate cyclase and activate a chain of events in the vasculature including smooth muscle cell relaxation, inhibition of platelet aggregation and neutrophil margination and regulation of gene expression. In SCD, the production of .NO appears to be chronically activated to maintain vasodilation, as indicated by low baseline blood pressure, decreased pressor responses to angiotensin II, renal hyperfiltration and a tendency for priapism. Plasma arginine levels drop precipitously during pain crises, indicating a possible demand for, or insufficient synthesis of, .NO. The mechanisms underlying blood flow deprivation, the associated pain and consequent tissue injury in SCD remain poorly understood. If the tissue ischemia that is a hallmark of SCD resulted solely from polymerized, sickled red cells, occlusion of predominantly small blood vessels would occur. In contrast, stroke in SCD results from occlusion of large and medium-sized arteries (internal carotid and middle cerebral arteries). Importantly, levels of sickled erythrocytes or dense cells do not correlate with painful episodes and other manifestations of vascular occlusion, inferring that morbidity is due to vascular functional defects that occur in response to sickling, rather than mechanical effects of sickling.

Increased oxidant production in the vasculature of SCD patients has been recognized for almost two decades. However, this disclosure reveals that the endogenous rate of production of superoxide (O₂.—) and hydrogen peroxide (H₂O₂) by human sickle red cells is not significantly increased. In contrast, elevated plasma and vessel wall xanthine oxidase (XO) and myeloperoxidase activity in SCD patients and SCD mice, and increased vessel wall O₂.— and H₂O₂ generation in SCD mice is observed. This is ascribed to the a) vessel wall binding of liver-derived circulating XO, released following repeated hepatic hypoxia-reoxygenation events, b) release and vessel wall binding of of neutrophil myeloperoxidase, and c) possible increased vessel wall expression of XO or other oxidases. This vascular inflammatory condition in SCD can induce O₂.— and H₂O₂ dependent inhibition of the salutary actions of .NO, while concomitantly yielding the potent and versatile reaction products, peroxynitrite (ONOO—) and nitrogen dioxide, oxidizing and nitrating species capable of further impairing vascular function. Thus, it is viewed that XO-derived reactive species impair nitric oxide-dependent systemic vascular function in SCD patients and contribute to the pathogenesis of acute sickle cell crises and end-organ damage. Therefore, a therapeutic regime to target and inhibit the XO-dependent production of O₂. and H₂O₂ should be effective in treating SCD patients by preserving .NO functions and endothelial dependent function in SCD patients.

### SUMMARY

This disclosure provides a method to inhibit the increases in levels of oxidants, namely superoxide and hydrogen peroxide, associated with impairment of vascular function in sickle cell disease and other disease states. Superoxide and hydrogen peroxide levels are decreased by inhibiting the activity of xanthine oxidase, a source of oxidant production in sickle cell disease, ischemia/repurfusion injury and other physiological processes. By inhibiting oxidant production by xanthine oxidase, nitric oxide levels are increased allowing resumption of normal vascular function.

### BRIEF DESCRIPTION FO THE DRAWINGS

So that the features, advantages and objects of the disclosure will become clear, are attained and can be understood in detail, reference is made to the appended drawings, which are described briefly below. It is to be noted, however, that the appended drawings illustrate preferred embodiments of the disclosure and therefore are not to be considered limiting in their scope.

FIG. 1A shows that vascular endothelial XO binding increases cellular rates of O₂.— production. After 3 hr incubation with XO at 37° C., cells were washed and assayed for rates of O₂.— production by cytochrome c reduction in the presence of 100 μM xanthine:

FIG. 1B shows endocytosis of cell-bound XO. Cells were incubated with ¹²⁵I-XO at 37° C., washed and treated with 0.5% trypsin. ¹²⁵I-XO was measured in both cell pellets (●) and supernatant (▪);.

FIG. 1C shows endothelial binding and transcytosis of neutrophil-derived myeloperoxidase (MPO). Neutrophils were activated by exposure to an inflammatory mediator, causing their binding to the vessel wall and subsequent release of MPO that can then serve as a vascular source of secondary oxidizing, nitrating, chlorinating and NO-consuming activities.

FIG. 2A shows prior exposure to XO inhibits endothelial-dependent relaxation. Incubation of aortic rings with XO (10 mU/ml) for 1 hr, followed by extensive washing, reduced vasodilation in response to acetylcholine in a heparin (10-1000 U/ml)- and allopurinol (10-250 μM)-reversible fashion;

FIG. 2B shows cholesterol feeding, shown to stimulate increased levels of circulating XO and vessel wall XO activity inhibits endothelial-dependent relaxation. Aortic rings from rabbits on a 1% cholesterol diet exhibit diminished vasorelaxant responses to acetylcholine. Pretreatment of rings with 1000 U/ml heparin to cause release of vessel wall-bound XO or 100 μM allopurinol partially restored endothelial-dependent relaxation;

FIG. 3 shows cell-bound XO inhibits .NO-dependent guanylate cyclase activation. Endothelial cells were cultured on Transwell filters and exposed to XO (10 mU/ml) for 3 hr and washed extensively. Filters were then transferred to dishes containing smooth muscle cells and incubated with xanthine (100 μM) and ionomycin (6.7 μM)±allopurinol for 15 min. cGMP was then determined by ELISA. (n=3);

FIG. 4A shows rates of O₂. release by HbA and HbS red cells. Rates of O₂. release for HbA and HbS red cellls are not significantly different. Values are the mean±SEM (n=3 to 9). Statistical analysis was by two-way ANOVA with Tukey post hoc test. *, p<0.05;

FIG. 4B shows aminotriazole mediated catalase inactivation by HbA and HbS red cells. These results indicate that rates of H₂O₂ release for HbA and HbS red cellls are not significantly different. Values at each time point represent mean±SEM with n=5;

FIG. 4C shows rates of HbA and HbS red cell nitric oxide consumption during hypoxic and normoxic conditions. These results indicate that net rates of oxygen radical production by HbA and HbS red cellls are not significantly different. Values represent mean±SEM (n=4 to 14). Statistical analysis was by two-way ANOVA with Tukey post hoc test. *, p<0.05 compared with basal;

FIG. 5A Shows Western blot analysis of plasma and liver XO in SCD mice; These results support the precept that liver-derived XO is released and increases levels of XO in the circulation;

FIG. 5B shows immunocytochemical analysis of xanthine oxidoreductase in C57B1/6J control and sickle cell mouse tissues. Descending thoracic aortic segments from knockout-transgenic SC mice display intense immunofluorescent staining for XO (red) that is associated with the endothelium and to a lesser extent, smooth muscle cells (L, lumen). Liver sections from SC mice show decreased xanthine oxidoreductase staining in the pericentral hepatocytes when compared to controls (CV, central vein). Nuclei were counter-stained with Hoechst in all experiments;

FIG. 5C shows hematoxylin-eosin staining of liver sections from control and sickle cell mouse tissues;

FIG. 6 shows O₂. production by C57B1/6J control and sickle cell mouse vessels. Values represent mean±SD (n=4). Statistical analysis was by two-way ANOVA with the Duncan's post hoc test. *, p<0.05 compared to control. +, p<0.05 compared to xanthine-treated sickle cell vessels. **, p<105 compared to control and all treated vessel groups.

### DETAILED DESCRIPTION

Oxygen Radical Formation and Tissue Injury

Aerobiosis permits efficient cell energy metabolism and concomitantly exposes organisms to reactive and potentially toxic oxygen byproducts. During normal cellular aerobic metabolism, about 98% of molecular oxygen is fully reduced to H₂O₂ by 4 e⁻ transfer at mitochondrial cytochrome c oxidase, with no release of partially-reduced intermediates. The remaining O₂ consumption includes 1 or 2 e⁻ reduction of O₂ to O₂. and H₂O₂ (1). Diverse cell components are responsible for O₂³¹ and H₂O₂ production. Membrane-bound e⁻ transport systems (mitochondrial respiratory chain, endoplasmic reticular cytochrome P450 system, the NADPH oxidase system of polymorphonuclear, PMN, cells) actively reduce O₂ to O₂. (2). Neutrophil-like oxidase(s) also serve as a key sources of reactive oxygen (3-5). Other proteins including hemoglobin, xanthine oxidase (XO) and .NO synthases are critical sources of O₂. and H₂O₂, with the spontaneous or enzymatically-catalyzed dismutation of O₂. also yielding H₂O₂ (1,6,7). Normally, endogenous tissue antioxidant defenses such as the superoxide dismutases (SOD), catalase, the glutathione peroxidase system and soluble or lipophilic scavengers (ascorbate, thiols, α- and γ-tocopherol, β-carotene) maintain intracellular concentrations of reactive oxygen species in the nM range or less (2). Diverse inflammatory mediators can activate oxidant production to overwhelm tissue antioxidant capabilities, facilitate target molecule reactions and cause toxicity via impairment of metabolic and structural elements of tissues. The singular oxidative characteristics of different cell types and tissues determines that a unique spectrum of oxidizing species will be produced at various tissue sites. At sub-μM rates of production (e.g., steady state concentrations in the 1-100 nM range), the influence of reactive species on modulating redox-sensitive cell signaling reactions will predominate over cytotoxic effects.

Xanthine oxidoreductase (XOR, EC. 1.2.3.2.) was first described in milk in 1934 and served as a model metalloprotein in the study of enzymatic redox reactions until two landmark discoveries occurred when xanthine oxidase (XO) (XOR is converted to XO by several mechanisms, discussed beow) was shown to be a) the first biological source of O₂. (34) and b) a key source of reactive oxygen species in tissue ischemia-reperfusion injury (35). Native XOR consists of 2 identical 130 kD subunits, each containing one molybdenum, one flavin adenine dinucleotide (FAD) and two Fe—S centers, and has a broad substrate specificity, serving to oxidatively hydroxylate reducing substrates (e.g., purines and aldehydes). Oxidative hydroxylation occurs at the purine center and then substrate-derived electrons are transferred via Fe—S centers to the flavin moiety. Xanthine oxidoreductase typically exists in cells as an NADH-producing dehydrogenase (XDH) that displays ˜10% partial oxidase activity (e.g., electrons are promiscuously transferred, both univalently and divalently, to solvated O₂ to yield O₂. and H₂O₂). Upon thiol oxidation, mixed disulfide formation or partial proteolysis, XOR is reversibly (moderate thiol oxidation) or irreversibly (extensive thiol oxidation, proteolysis) converted to XO (36). Upon conversion to XO, reduction of O₂ to O₂. and H₂O₂ occurs, typically in a 1:2 ratio of O₂.⁻:H₂O₂. The release of XOR into the systemic circulation results in rapid XDH to XO conversion (<1 min) via thiol oxidation (37). The intracellular XDH to XO conversion that can occur during metabolic stress is due to both thiol oxidation and proteolysis. However, these conversions are not a requisite for XOR-derived oxidant production, due to the partial oxidase activity of XOR (36, 38). In the remainder of this disclosure, XO will be understood to include the activities of XOR and XDH as would be understood by one of ordinary skill in the art.

The physiologic functions of XO are not extensively characterized, but include a) purine metabolism, b) oxidant and, paradoxically, antioxidant production, c) drug metabolism and d) signal transduction. The primary physiologic role of XO is to function as the rate-limiting enzyme in purine degradation, yielding xanthine from hypoxanthine and uric acid from xanthine. The evolutionary loss of urate oxidase, which catabolizes uric acid to allantoin, makes uric acid the terminal product of purine metabolism in humans (39). Therefore, the inherent, endogenous property of XO is not to generate cytotoxic oxidants, but rather to participate in physiologic processes, in contrast to the excess production of XO-generated reactive species experienced during pathologic states.

Sources of Xanthine Oxidase

Circulating XO can be derived from several sources, including the liver and the intestine, which contain the greatest tissue specific activity of XOR (76), the vascular endothelium and from phagocytic cells during inflammatory events. Several studies have shown that even minimal hepatocellular damage, such as procedures that render the liver ischemic, increases XO levels in the circulation of humans (77, 78). Circulating XO increases 2-fold in patients undergoing thoracic aorta aneurysm repair, a procedure that renders liver, intestine, and all distal tissues ischemic (78). Subsequent reperfusion of tissues below the renal artery did not increase circulating XO, suggesting that the liver and gut were the sources of XO. Circulating XO is also elevated in adult respiratory distress (79) and kidney disease (77). Vascular endothelial cells may also be a source of circulating XO, since interruption of the blood supply to an upper limb of human patients undergoing an orthopedic procedure increased plasma levels of XO (80).

These clinical observations have also been confirmed in animal models and in vitro studies. A variety of conditions, including organ ischemia-reperfusion, sepsis, burns, acute viral infection and hemorrhagic shock all induce release of XO from liver and gut into the plasma of rat and rabbit (81-88). In vitro studies indicate that it is likely that both hepatocytes and vascular endothelium are significant sources of circulating XO, since exposure of freshly isolated hepatocytes (89) or cultured endothelial cells to hypoxia results in release of XDH+XO into the media (75). These reports all indicate that XO plasma half-life may be hours, not minutes as previously suggested (37). Importantly, tissue release of XO into the circulation may occur in the absence of disrupted metabolic homeostasis, since up to 8% of plasma IgG from healthy humans is immunoreactive with XO and is present as a circulating immune complex that is partially inhibitory to XO activity (36). Thus, when present in excess, circulating XO can serve as an intravascular locus of O₂. and H₂O₂ which can a) directly cause tissue injury, b) generate secondary species, c) deplete tissue antioxidants, and d) activate secondary inflammatory responses by generating chemotactic oxidized products (90).

Table 1 shows the level of XO activity in the plasma of different species in response to various vasculopathies. Although there is a difference in the activity of XO (under both basal and pathological conditions), the various vasculopathies all resulted in the release of active XO into the plasma. This released XO is, therefore, available for binding to and uptake by cells lining the vascular walls. Plasma activity of XO is elevated in rabbit animal models of hemorrhage and aortic occlusion that result in hepatic ischemia/reperfusion and the release of XO from hepatocytes (81-84, 87, 224). In addition to hepato-enteric injury models, elevations in plasma XO activity have been observed in systemic processes such as rabbits fed an atherogenic diet (226). Recurrent vascular occlusive crises in patients with sickle cell disease have also been postulated to be the result of XO-mediated oxidative stress (227). Venous plasma XO activity is significantly elevated in SCD patients, even when not in crisis, when compared to plasma obtained from volunteers with HbAA. The presence of XO in human plasma and the low specific activity of XO in human endothelium lend significance to the concept that circulating XO can bind to and concentrate both on and in vascular cells

TABLE 1

| Species | Condition | Plasma XO Activity (μU/ml) |
|---|---|---|
| Rat | Control | 500 ± 3 |
| | Hemorrhage | 1200 ± 17* |
| Rabbit | Control | 40 ± 12 |
| | Aortic Occlusion | 1085 ± 316* |
| Rabbit | Control | 46 ± 4 |
| | Chol-fed | 113 ± 15* |
| Human | Control | 0.9 ± 0.3 |
| | Sickle Cell | 3.3 ± 0.9* |
| Human | Pre Aortic Xclamp | <1.0 |
| | Post Reperfusion (1 min) | 65 ± 10* |
| | Post Reperfusion (20 min) | 15 ± 3* |

Summary of the circulating XO activity in different species and vasculopathics. *represents p < 0.05 vs. control plasma. Data are mean ± SEM. n = 6 (hemorrhage), 8 (aortic occlusion), 10 (cholesterol feeding), 6 (sickle cell), 13 (liver transplant)

Diverse proteins, including XO, are synthesized and secreted by tissue parenchymal cells (i.e. liver) into the circulation where they can bind to vascular endothelium (91, 92). Alternatively, enzymes can be synthesized by the vascular cells and then secreted to the vascular cell surface to function as ectoenzymes. These proteins can bind in a saturable, high affinity manner to the cell surface. They include lipoprotein lipase, vitamin K-dependent coagulation proteins, prothrombin, diamine oxidase, cell adhesion molecules, proteases, antiproteases, heparin-binding growth factors and the antioxidant enzyme extracellular CuZn superoxide dismutase (EC-SOD) (93-100). This cell binding is mediated primarily by glycosaminoglycans (GAGs), anionic polysaccharides consisting of repeating disaccharides, often sulfated in the carbohydrate backbone. In most cases, we lack detailed understanding of the relationship between GAG structure, enzyme binding affinity and GAG modulation of bound enzyme function. Because of their strong polyanionic nature, GAGs may also bind circulating molecules that originate from remote tissues, such as XO produced by cells of the liver, concentrating them at the cell surface with significant metabolic and pathologic implications.

The high affinity binding of XO to glycosaminoglycans (GAGs) facilitates XO interactions with the vessel wall. Addition of purified cultured endothelium to XO containing medium results in concomitant loss of XO from the medium and avid cell-XO association. We also observed that a) heparinization of rats subjected to ischemia-reperfusion injury via hemorrhagic shock had greater plasma levels of circulating XO (84) and b) perfusion of isolated lungs with effluent from ischemic liver increased lung XO activity 14 fold (100). These data all imply that XO was binding to cell GAGs and stimulated the in vitro binding of bovine milk XO to Sepharose 6B-conjugated heparin (HS6B). This binding interaction has a K_{d} of 40-180 nM, similar to the binding affinities of several known vascular cell GAG-binding proteins. Kinetic parameters of heparin-bound XO changed in a manner reflecting altered enzyme affinity for substrates and inhibitors. Interestingly, greater concentrations of CuZn SOD were required to scavenge the O₂.— produced by GAG-bound, versus soluble, XO. Finally, the oxidative self-inactivation of XO was impaired (228). In aggregate, these data are relevant to pathologic oxidative processes in SCD because they indicate that XO released from a remote organ (e.g., liver) could bind to the systemic vasculature and to impair cell signaling and hemodynamics.

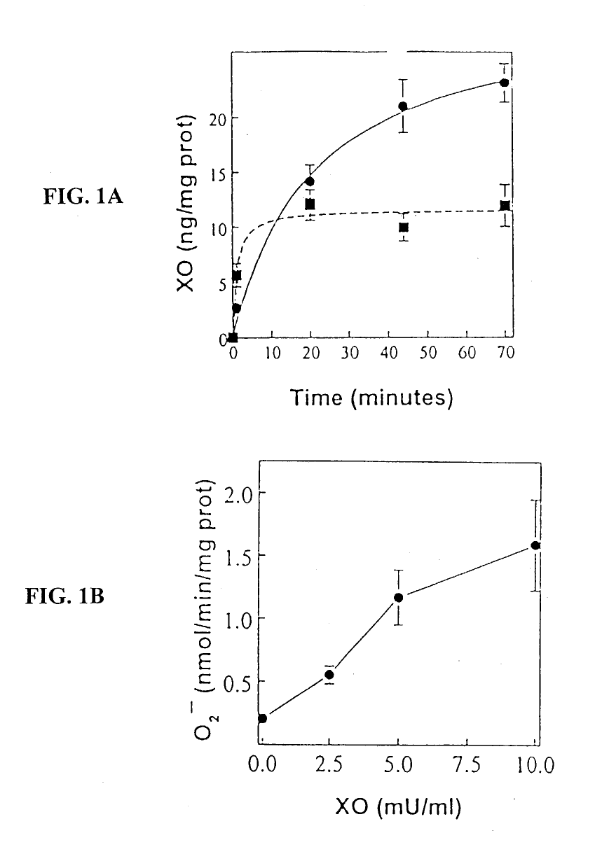

As previously reported by the inventors, XO binds in a specific and high affinity fashion to cultured endothelial cells with a K_{d}=6 nM, comparable to the K_{d} observed for XO binding to HS6B (229). XO initially binds through interactions with cell surface sulfated GAGS, since bound XO is partially displaced from the endothelium by heparin and pretreatment of cells with chondroitinase limits XO binding. Neither heparinase nor heparitinase prevented XO association with endothelium. The partial displacement of XO binding by heparin is due to the polyanionic character of heparin and its ability to bind to cationic motifs of XO, in turn competing for XO binding to GAG-containing cellular proteoglycans. This binding increased cell XO activity, 10-fold and resulted in increased cellular O₂.—, production (FIG. 1A).

The cell-associated XO initially localized to a trypsin-sensitive compartment, however, catalytically-active XO rapidly translocated to a trypsin-insensitive compartment via transcytosis or endocytosis (FIG. 1B). These data demonstrate the efficacy with which XO binds to endothelial cells, resulting in enhanced rates of both cell surface and intracellular oxidant production. The ability of circulating XO to bind to vascular cells may explain the utility of XO inhibitors in the clinical protection of remote organs from oxidant-induced injury. Another important implication of this data is that bound XO may thus be one of the apparent sources of the “inducible” NAD(P)H oxidase activity observed in many vascular cells exposed to inflammatory conditions.

In addition to XO binding to endothelial cells, myeloperoxidase (MPO) is also capable of cellular binding internalization and production of reactive oxygen species. Neutrophils that have been activated by exposure to an inflammatory mediator bind vessel walls and release MPO. The MPO then binds the vessel wall and may ultimately become internalized. In this manner, MPO can serve as a vascular source of secondary oxidizing, nitrating, chlorinating and .NO-consuming activities.

The above is consistent with the observation that the XO specific activity of lung (100) and myocardium (101) is markedly increased following hepatoenteric ischemia/repurfusion. The myocardial and pulmonary injury associated with hepatoenteric I/R is also attenuated by inhibition of XO, implicating circulating XO in remote tissue damage (100, 101). In addition, the XO inhibitor allopurinol, which exhibits no direct antioxidant properties at pharmacologic concentrations (70), displays tissue-protective actions in organ systems low in or devoid of detectable endogenous XO activity implying allopurinol is inhibiting XO in these tissues that was produced at a remote location. For example, allopurinol attenuated both rabbit and clinical myocardial ischemia-reperfusion injury, in spite of several reports revealing that rabbit and human heart have low to undetectable levels of XO under normal physiological conditions (71-73).

These observations have profound implications for XO-GAG interactions by inferring that XO can be released from metabolically-stressed cells, bind to cell surface GAGs as an external source of oxidant production, and ultimately become internalized by endocytosis/transcytosis to become an intracellular locus of oxidant production. Also, endothelial transcytosis of XO can result inn vessel wall/cell matrix deposition of this source of oxidative stress. Thus, during diverse pathologic processes XO, released into plasma from cells replete in XO specific activity, can circulate to remote sites and bind to target tissues low in or devoid of XO activity. Cell-bound XO may then be concentrated at the cell surface or interstitial matrix where oxidant products can more readily react with cellular target molecules, disrupt vascular function and acquire limited access to or reactivity with inhibitors. These data also imply that effective scavenging of intravascular O₂. must often take place at the cell surface and/or intracellularly. It therefore becomes critical to understand the tissue distribution of XO and underlying mechanisms of cell injury that are mediated by a source of reactive species widely implicated in various pathological processes. Understanding of the potential for bidirectional trafficking of XO and its ability to act as a paracrine agent will be critical for appreciating its vascular signaling/injury mechanisms.

Xanthine Oxidase O₂. Production Affects Normal Physiology

Of significance are the recent clinical studies reporting strong correlations between plasma uric acid levels and the risk of stroke, congestive heart failure and renal dysfunction in patients with vascular disease (115-119). The elevated uric acid levels observed in these clinical studies do not merely correlate with impaired renal function (eg, decreased creatinine and/or uric acid clearance), but are indicative of enhanced XO catabolism of purines and the presumed generation of reactive oxygen species that may be mediating direct vascular injury and the impairment of .NO signaling. Critically, and in the context of the present hypothesis, plasma uric acid levels are significantly elevated in young and adult sickle cell patients of both sexes, in spite of increased rates of uric acid clearance in SCD patients (120-122). This implies enhanced XO activity and production of reactive species in SCD patients.

Nitric Oxide and Vascular Function

The free radical gas .NO is produced following nitric oxide synthase (NOS) activation by inflammatory (iNOS) and vasoactive/neurotransmitter (eNOS; nNOS) mediators, and plays a critical role in multiple aspects of vascular function (132). Catalytic activity and immunoreactivity of both inducible and constitutive forms of iNOS occurs both in vivo and in cultures of vascular cells, with iNOS predominating in smooth muscle cells and infiltrating leukocytes, while eNOS is localized to endothelial cells and cardiac myocytes (133). Evidence of vascular .NO production comes from the detection of the .NO metabolites NO₂. (nitrite) and NO₃— (nitrate) and S-nitrosothiol derivatives of albumin and hemoglobin (RSNO) (134, 135). The increased .NO production by vascular cells exposed to inflammatory mediators infers participation in host defense and free radical-mediated tissue injury (136). Nitric oxide may also be produced at low levels by human inflammatory cells, including neutrophils and monocytes/macrophages (137-143). Macrophage-derived .NO serves an immunomodulatory role, with the pathogen-killing activity revealing L-arginine dependence, NOS/.NO inducibility and concomitant production of the .NO oxidation products NO₂— and NO₃— (145, 146). An important (if not principal) role for .NO has been established in macrophage tumoricidal activity and the killing of invading microbes and parasites (147). Many forms of acute and/or chronic vascular inflammatory reactions display enhanced production of .NO that can contribute to tissue injury, with NOS expression and plasma NO₂—+NO₃— levels elevated (132, 146, 147). Although .NO clearly modulates diverse homeostatic and pathophysiological pathways, the non cGMP-dependent mechanisms by which .NO acts are only partially understood.

The importance of .NO in the regulation of coronary and systemic vasodilator tone has been demonstrated experimentally by inhibiting its synthesis. Thus, N^{G}-monomethyl-L-arginine (L-NMMA), which competes with L-arginine as the substrate for nitric oxide synthase but cannot be oxidized to form .NO increases basal systemic and coronary vascular resistance and blunts the vasodilator response to the endothelium-dependent vasodilator agonists acetylcholine and bradykinin. Intracoronary infusion of L-NMMA caused coronary epicardial constriction and reduced coronary blood flow as a result of microvascular constriction in patients without atherosclerosis or its risk factors, indicating that .NO contributes to basal coronary vasodilator tone and blood flow (188). In contrast, patients with coronary artery disease or risk factors for atherosclerosis showed minimal vascular responses to L-NMMA, suggesting deficient .NO release from the coronary endothelium in these conditions. .NO also contributes to regulation of vascular tone in the systemic circulation. When forearm blood flow is measured by strain gauge venous-occlusion plethysmography, before and after inhibition of .NO synthesis in the forearm with infusion of L-NMMA into the brachial artery, L-NMMA significantly reduced forearm blood flow, a vasoconstrictor effect, indicating the important contribution of .NO to the vasodilator tone of forearm arteriolar resistance vessels (189). This response to L-NMMA was reduced in subjects with hypertension, diabetes, and hypercholesterolemia, suggesting reduced endothelium-derived .NO in these conditions (189). Indeed, recent reports suggest that dysfunctional vascular .NO production, characterized by a paradoxical vasoconstrictor response to acetylcholine (an agonist that normally increases blood flow by stimulating endothelial .NO production) predicts future cardiac ischemic events (190; 191, 192). Thus a deficiency in endothelial production of .NO is increasingly being recognized as an underlying pathogenic mechanism in vascular diseases.

A number of models of vascular injury reveal that endogenous .NO biosynthesis or exogenously-added sources of .NO often inhibits oxidant-dependent damage at both molecular and tissue structural/functional levels. Many, if not all of these studies, have inflammatory injury as a common denominator. It was initially reported that PMN-derived O₂.— is “inactivated” or “scavenged” by .NO (172). However, .NO was added at extra-physiological concentrations and the report failed to consider that peroxynitrite, ONOO—, an even more potent oxidant, was the product of the “scavenging” reaction. .NO can also directly inhibit the NADPH oxidase of PMN cells, but again only at non-biological concentrations (173). At lower concentrations, .NO inhibits leukocyte adhesion to vascular endothelium, attenuates PMN-dependent loss of microvascular barrier function and inhibits platelet aggregation, all components of inflammatory vascular injury (174-176). The protective effects of .NO towards in vivo models of reperfusion injury, when .NO is administered as a bolus of an .NO donating drug, are thus often ascribed to .NO inhibition of inflammatory cell margination and function (177-181). Underlying mechanisms include both acute events and more delayed processes involving regulation of integrin gene expression. Acutely, .NO administration to reperfused ischemic tissues will result in stimulation of vessel wall, circulating platelet and neutrophil cGMP levels. This results in increased blood flow and oxygen delivery to tissues, as well as alterations in shear forces on the vessel wall, critical for regulating vascular-inflammatory cell interactions and secondary gene expression events (182). The translocation of P-selectin to the platelet surface and/or the function of P-selectin is inhibited by .NO as well, resulting in attenuation of platelet aggregation and neutrophil margination. Mast cell degranulation is inhibited by .NO, limiting the release of proinflammatory mediators such as histamine and platelet activating factor (180). Enzymatic and autocatalytic lipid oxidation is also potently inhibited by .NO (164, 183, 184), often resulting in attenuated inflammatory mediator production.

Nitric Oxide and Sickle Cell Disease

SCD is a genetic disease characterized by a mutant hemoglobin β-globin subunit with a glutamic acid to valine substitution at the β-6 amino acid. Upon deoxygenation, polymerization of HbS occurs and sickle erythrocytes acquire altered rheological properties (8). Altered red cell-tissue interactions induce increased vascular endothelial “activation” via poorly understood mechanisms. This inflammatory-like activated state of endothelium is manifested by elevated expression of Fc receptors and the integrins ICAM-1, VCAM-1 and P-selectin, all of which contribute to increased endothelial association of platelets and neutrophils (9-14). In addition, vascular levels of “activated” circulating endothelial cells, pro-inflammatory cytokines, platelet activating factor, C-reactive protein, and angiogenic stimuli are increased (15-17).

Recent clinical data suggest that patients with SCD also suffer from impairment of endothelial production of .NO. Indeed, several investigators have now reported that NO₂⁻+NO₃⁻ levels and L-arginine are depressed in patients with SCD, particularly during vaso-occlusive crisis and the acute chest syndrome, and that these levels vary inversely with pain symptomatology (193, 194, 195, 196). These data suggest that dysfunctional vascular endothelium and decreased .NO production and/or bioavailability may contribute to the clinical events suffered by patients with SCD. Reduced NO₂⁻+NO₃⁻ levels are consistent with reduced endothelial .NO generation, with subsequent reductions in the NO₂⁻+NO₃⁻ metabolites of .NO reactions with hemoglobin and oxygen. Furthermore, L-arginine levels are reduced in sickle cell individuals, particularly during vaso-occlusive crisis and the acute chest syndrome, suggesting that substrate deficiency may explain the low levels of NO₂⁻+NO₃⁻ (197). However, large doses of L-arginine do not result in increased .NO generation in clinically able volunteers with SCD.

Low baseline blood pressures (18) and decreased plasma arginine levels (19) indicate that .NO production is chronically activated to maintain vasodilation in SCD. Also, decreased pressor responses to angiotensin II (20), renal hyperfiltration (21), a tendency for priapism (22) and elevated plasma nitrite and nitrate (NO₂⁻+NO₃⁻) levels occur in SCD (23). During vaso-occlusive crisis (VOC), an increased metabolic demand for arginine and an inverse relationship between pain indices and plasma NO₂⁻+NO₃⁻ levels occur (24, 25). Finally, therapeutic benefit has been observed in SCD patients receiving inhaled .NO or the drug hydroxyurea. Interestingly, hydroxyurea not only induces expression of anti-sickling fetal Hb, but is also metabolized to .NO via oxidative deamination (26, 27). Increased rates of production of reactive oxygen species have been proposed to contribute to impaired .NO signaling in SCD. HbS red cells generate O₂.—, H₂O₂, .OH and lipid oxidation products (LOOH, LOO.) (28). Furthermore, decompartmentalization of the redox-active metal iron occurs in HbS red cells (29). SCD mice show increased tissue lipid oxidation, .OH or ONOO⁻-dependent aromatic hydroxylation and, during hypoxia, increased conversion of liver and kidney XOR to XO (30, 31). Since .NO reacts at diffusion-limited rates with O₂. and lipid peroxyl radicals (LOO.) to produce peroxynitrite (ONOO—) and nitrated lipid species [LNO₂, L(O)NO₂], the anti-platelet and PMN actions of .NO can be oxidatively “inactivated” while at the same time yielding secondary bioactive products (32, 33). The impaired vascular function and inflammatory activation of SCD vessels can thus be a consequence of the stimulation of oxygen radical-mediated consumption of .NO and the production of secondary reactive species (e.g., H₂O₂ or ONOO—) that can also impair vascular function.

The above discussion has outlined a potential interaction of O₂. produced by XO and .NO and its effect on vascular function, in particular, on vascular function in patients not only with SCD, but also with athrosclerosis. Briefly, it was shown that plasma and vessel wall XO activity, normally low in humans, can be significantly increased during diverse pathogenic events via XO binding to vascular cells by interaction with cellular GAG chains and endocytosis of circulating XO, and that the reactive species derived from cell-bound XO and MPO can modify blood vessel, pulmonary and cardiovascular function. Furthermore, episodes of hypoxia-reoxygenation associated with SCD can lead to the hepatic release of XO into the circulation, where XO can then bind avidly to vessel luminal cells and impair vascular function by creating an oxidative milieu, which catalytically consumes cellular .NO. Taken together these observations suggested the testing of pharmacologic strategies to inhibit the production of reactive oxygen species produced by XO could lead to a valuable therapeutic regime for those individuals suffering from SCD.

Allopurinol is an agent widely used in treatment of hyperuricemic states such as gout. Allopurinol and its primary metabolite, oxypurinol, and other pyrazole derivatives inhibit hyperuricemia by inhibiting the enzyme XO, which converts hypoxanthine to xanthine, which is further converted into uric acid. Oxypurinol has been reported to have increased water solubility as compared to allopurinol. In this specification, allopurinol shall be understood to refer to oxypurinol and all other metabolites of allopurinol that are active XO inhibitors, as well as chemical derivatives of allopurinol. The term “chemical derivative” refers to allopurinol that contains additional chemical moieties or that are not normally a part of the base allopurinol molecule. Additional compounds that may be used to inhibit allopurinol activity are described in U.S. Pat. No. 6,191,136 (which is incorporated by reference herein).

Xanthine Oxidase Impairs Vascular Signaling

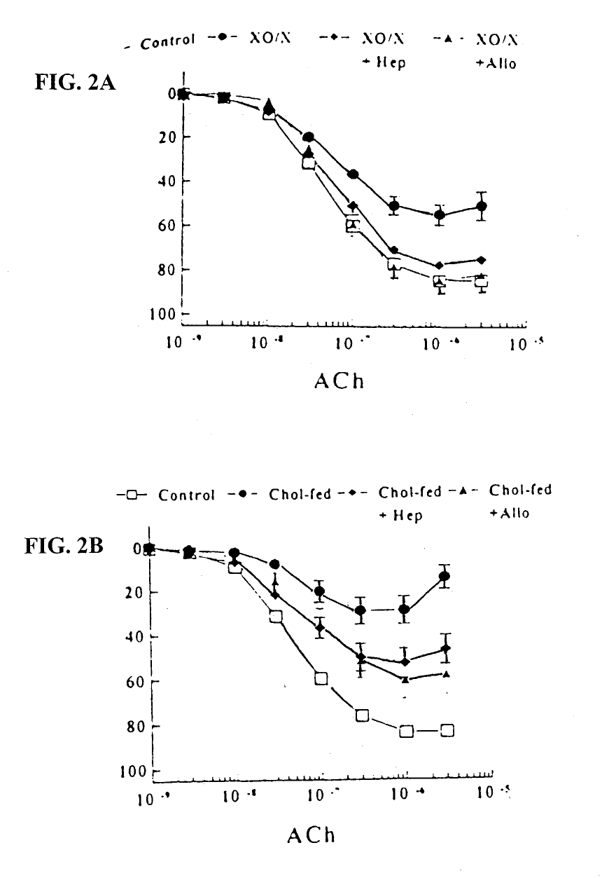

Increased cell-associated XO can impair endothelial-dependent vascular signaling. When isolated aortic ring segments are incubated with XO for 1 hr and then extensively washed to remove unbound XO, endothelial-dependent relaxations in response to acetylcholine are diminished when the XO substrate xanthine is present in the buffer (FIG. 2A). Inhibition of endothelial-dependent relaxation is abrogated by post-washing treatment of the aortic rings with the XO inhibitor allopurinol, as well as heparin, which inhibits and/or competes for XO binding to endothelial cell GAGs. This ex vivo model of vascular dysfunction reinforces observations made in a model of atherosclerosis. Rabbits placed on a cholesterol-enriched diet for 6 weeks demonstrate markedly abnormal responses to acetylcholine that are significantly reversed by treatment with heparin or allopurinol (FIG. 2B) (226). These data imply a role for vascular cell-bound XO in mediating abnormnal endothelial-dependent relaxation in atherosclerosis. Importantly, there appears to be sufficient substrate in the vasculature for XO to catalyze elevated oxidant production, without the need for exogenously added purine.

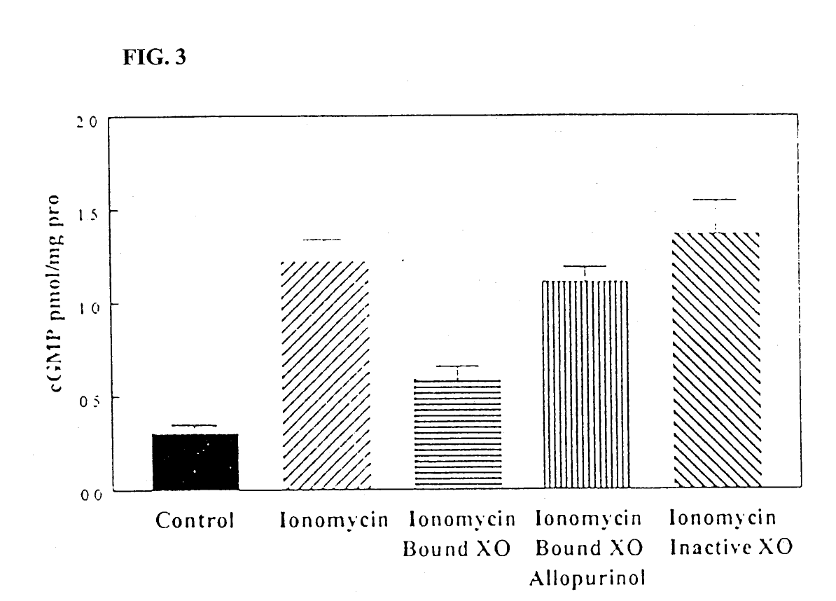

Endothelial cell-bound XO inhibits .NO-dependent cell signaling. Bovine aortic endothelial cell (BAEC) monolayers having bound and potentially internalized XO manifested an impaired ability to support ionomycin-induced, .NO-dependent cGMP formation by adjacent smooth muscle cells. This inhibition of .NO-mediated cGMP formation was allopurinol-reversible and did not occur when catalytically inactive XO was substituted (FIG. 3).

One mechanism that can account for XO-induced decreases in endothelial-dependent relaxation and impairment of .NO-mediated cGMP formation in smooth muscle cells is the diffusion-limited reaction of O₂.— with .NO to yield ONOO—, a less potent stimulus of smooth muscle cell cGMP formation than equimolar doses of .NO (230). Also, XO can serve as a source of H₂O₂ that in turn will support the oxidative reactions of myeloperoxidase (NO consumption and the generation of chlorinating, nitrating and oxidizing species). Another potential mechanism explaining XO inhibition of endothelial-dependent relaxation and cGMP formation may be oxidant-induced changes in the expression and/or activity of eNOS, especially during more chronic exposure to reactive oxygen species. Bovine aortic endothelial cells (BAEC) were exposed to the redox-cycling quinone 2,3-dimethoxynapthoquinone (DMNQ), which generates controllable and low rates of O2.— and H₂O₂. Following treatment with DMNQ for 2 hr, the capacity of endothelial cell lysates to oxidize arginine to citrulline was used to indicate eNOS activity. Additional cells were incubated with 10 mU/ml XO for 1 hr, extensively washed and 100 μM xanthine was added during a 1 hr post-incubation period. Table 2 demonstrates the dose-dependent reduction in eNOS activity following endothelial cell exposure to either DMNQ or XO. These conditions did not result in detectable cell necrosis, apoptosis or lysis after 24 hr of monitoring.

TABLE 2

| Condition | NOS activity (pmol/min/mg prot) |
|---|---|
| Control | 0.93 ± 0.28 |
| DMNQ 5 μM | 0.75 ± 0.09* |
| DMNQ 50 μM | 0.49 ± 0.60* |
| Bound XO | 0.19 ± 0.02* |

Non-cytotoxic levels of oxidant stress reduce eNOS activity. BAEC were incubated with DMNQ for 2 hr or XO for 1 hr, washed extensively, then incubated with xanthine for 1 hr at 37° C. Cells were harvested and NOS activity measured in the cell lysates. (n = 3, mean ± SEM, *p < 0.05)

Xanthine Oxidase Generated O₂. Can Impair .NO-Dependent Vascular Signaling in Sickle Cell Disease

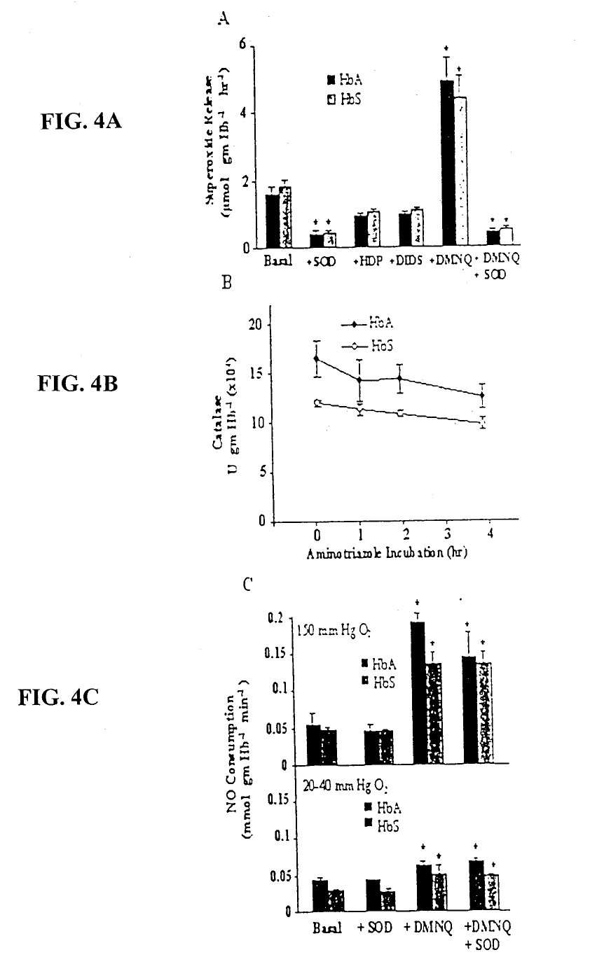

If red blood cells from sickle cell patients produce increased amounts of O₂. and H₂O₂ as compared to red blood cells from healthy donors, then local depletion of .NO might be explained on this basis. However, endogenous rates of O₂.— release under normoxic (150 mm Hg O₂, pH 7.4) conditions were not significantly different in human HbS vs HbA-containing red cells (FIG. 4A). The mean Hb content of HbA and HbS red cell preparations was similar. DMNQ (100 μM) was added to stimulate red cell O₂.— production. Preincubation of cells with the metal chelator HDP (0.5 mM) induced ˜36% decrease in O₂.—, suggesting that cellular Fe-dependent reactions partially contributed to cell O₂.— production. When cells were pretreated with a stilbene sulfonate chloride-bicarbonate exchange protein inhibitor (DDS, 200 μM), both HbA and HbS red cells showed ˜35% decrease in rates of cytochrome c reduction, indicating that some extracellular O₂.— was released through red cell anion channels. The similar slopes of the time course of amnotriazole (ATZ)-dependent red cell catalase inactivation in HbA and HbS red cells also revealed that steady state H₂O₂ levels were not significantly different in SC red cells, with calculated H₂O₂ concentrations of 3.11±2.61 pM and 4.9±2.25 pM, respectively, at 150 mm Hg O₂, pH 7.4 (FIG. 4B).

Accepting that *NO reacts at almost diffusion-limited rates with O₂.— (25), the relative rates of red cell .NO consumption was utilized to probe for differences in both intra-and extracellular O₂.— (and possibly LOO—) production by HbA and HbS red cells. Nitric oxide consumption, measured during both normoxic (150 mm Hg O₂) and sickling-inducing hypoxic (20-40 mm Hg O₂) (FIG. 4C) conditions, was not significantly different in HbA vs. HbS red cells. Addition of extracellular CuZn SOD (100 U ml⁻¹) did not impact on the rate of .NO consumption, while stimulation of cell O₂. generation by DMNQ (100 μM) significantly increased rates of red cell .NO consumption. The presence of endogenous lipid hydroperoxides was undetectable in both HbA and HbS RBC membranes. When membrane oxidation was stimulated by the addition of OONO—, HbS red cell membranes showed greater tendency to undergo lipid peroxidation (not shown). These data show, in contrast to previous reports, that red cells from SCD patients are not an endogenous free radical “sink” for the pathologic consumption of .NO.

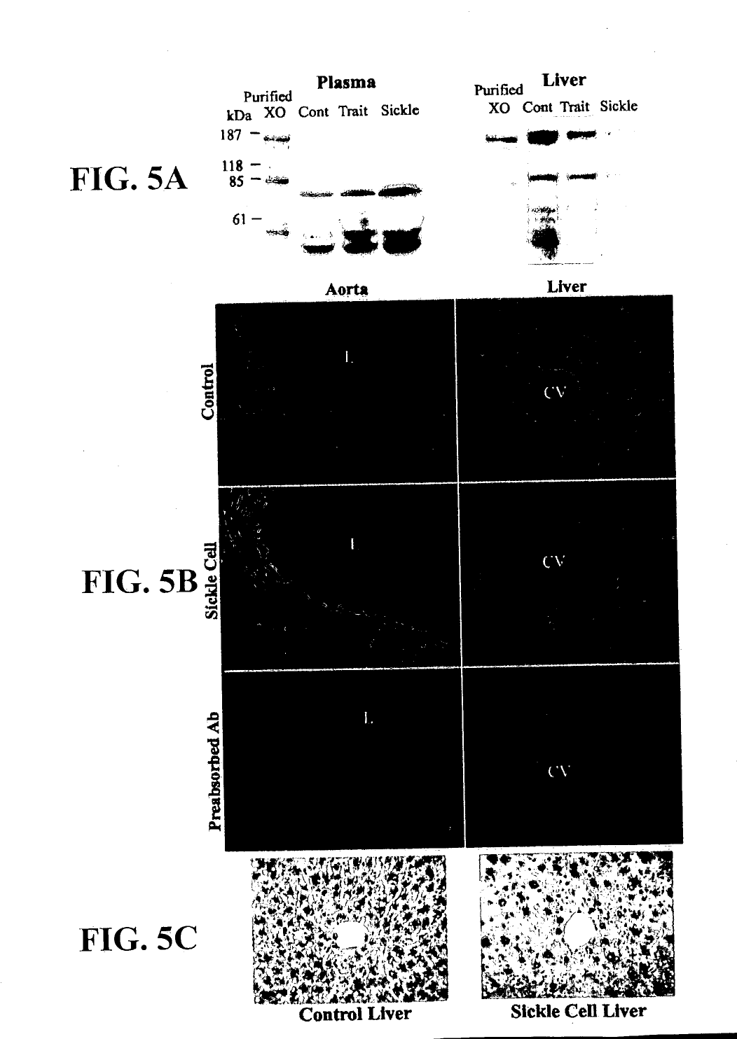

While the red blood cells of SCD patients do not produce excess amounts of O₂., the catalytic activity of XO was significantly increased in the plasma of SCD patients vs. controls. This also occurred in the plasma of our knockout-transgenic mouse model of SCD that exclusively expresses human HbS in the murine RBCs. The observed increase in plasma XO activity in knockout-transgenic SCD mouse was accompanied by a decrease in liver XO activity and an increase in plasma alanine transferase (ALT) activity (Table 3). Western blot analysis of plasma and liver XOR revealed increased plasma and decreased liver XOR protein content in knockout-transgenic SCD mice compared to control or knockout-transgenic sickle cell trait mice, that synthesize both human β^{S} and β^{A} (FIG. 5A). XOR which is rapidly converted to the XO in plasma, revealed immunoreactive 20 kDa, 40 kDa and 85 kDa proteolytic fragments upon western blot analysis. Immunocytochemical localization of XO in aorta and liver (FIG. 5B) of SCD mice showed increased vessel wall and decreased liver XO immunoreactivity, with XO concentrated on and in vascular luminal cells. Hematoxylin-eosin staining of liver of knockout-transgenic SCD mice reveals extensive hepatocellular injury associated with pericentral necrosis. Sickled erythrocytes were also observed in intrahepatic sinusoids (FIG. 5C).

TABLE 3

| | Enzyme Activity | |
|---|---|---|
| Measurement | Control | SCD |
| Human | | |
| Plasma XO (mU/ml) | 0.89 ± 0.3 (15) | 3.30 ± 0.9* (18) |
| Mouse | | |
| Plasma XO (mU/ml) | 2.2 ± 0.26 (13) | 5.6 ± 1.5* (15) |
| ALT (mU/ml) | 24.2 ± 2.3 (10) | 270.5 ± 24.5* (12) |
| Liver XO (mU/gm tissue) | 53.9 ± 7.8 (6) | 18.6 ± 4.4* (6) |
| Aortic XO (mU/mg protein) | 0.29 ± 0.01 (3) | 0.46 ± 0.03 (4) |

*p < 0.05 from control

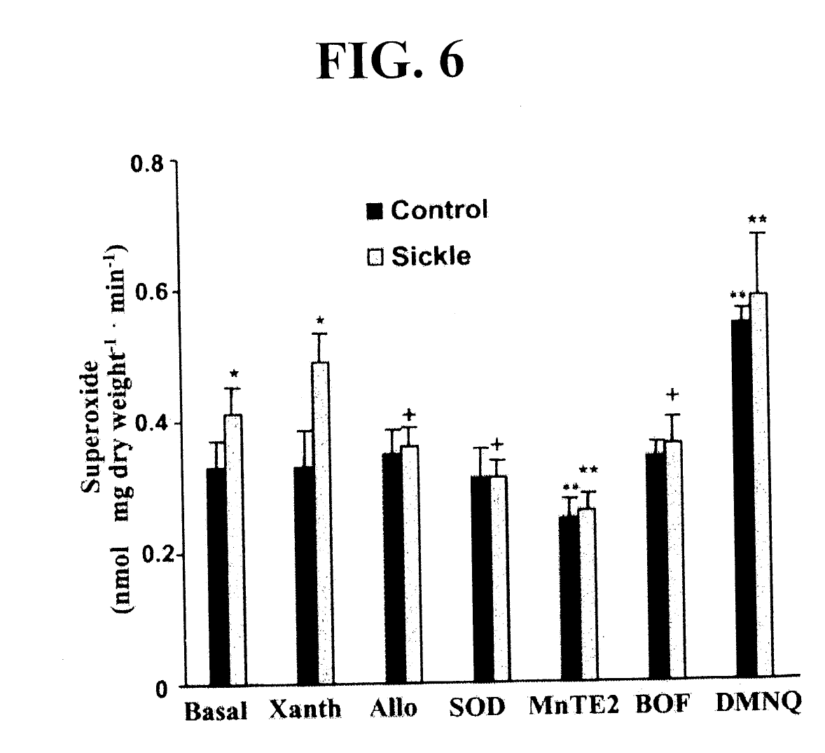

As is shown in Table 3, the catalytic activity of XO is significantly increased in the aorta of SCD mice with a parallel increase in XOR protein as observed by western blot analysis (FIG. 5A). Basal rates of O₂. production were measured by coelenterazine-enhanced chemiluminescence and was significantly increased in the aorta of SCD mice (FIG. 6). In SCD, but not wild type mouse vessels, rates of O₂.— production were enhanced by addition of xanthine and returned to basal rates when vessels were pretreated with CuZn SOD (30 U/ml), allopurinol (100 μM) or the XO inhibitor BOF-4272 (25 μM). Pretreatment of aorta with the membrane-permeable metalloporphyrin SOD mimetic MnTE-2-PyP (50 μM) significantly decreased rates of detectable O₂.— production, while DMNQ (100 μM) addition as a positive control significantly enhanced rates of O₂. production by both control and SCD mouse vessels. Additionally, addition of either the membrane-permeable metalloporphyrin SOD mimetic MnTE-2-PyP (50 μM) or allopurinol (50 μM) to vessel rings prepared from sickle cell mice resulted in reversal of impaired vessel relaxation in response to NO-dependent stimuli.

Taken together, these results indicate that reactive species impair .NO-dependent systemic vascular function in SCD, and that inhibitors which block the production of these reactive species are prime candidates for therapeutic agents aimed at sickle cell disease. The above results show that allopurinol is capable of reversing XO-mediated inhibition of acetylcholine-induced relaxation in endothelial cells (see FIGS. 2 and 3). Although not limiting the disclosure to a specific mechanism of action of allopurinol, the data suggest that allopurinol inhibits the activity of XO, decreasing the production of O₂. and H₂O₂. As a result, more .NO is available to stimulate smooth muscle cell relaxation through activation of cGMP-dependent signaling events. Also as a result, normal endothelial dependent vascular functions are restored in individuals with SCD. From this, a concomitant improvement in adverse pathophysiological sequelae of sickle cell disease will be expected, including painful episodes, acute chest syndrome, renal dysfunction.

Pharmaceutically useful compositions comprising allopurinol (or a pharmaceutically acceptable salt thereof) or other compounds of the disclosure may be administered either alone or in combination with modulating compounds, and may be formulated according to known methods such as by the admixture of a pharmaceutically acceptable carrier. Modulating compounds may be defined as any compound that modulates the activity of either allopurinol and/or oxypurinol. In one embodiment, allopurinol may be a modulating compound for oxypurinol administration, or vice versa. Examples of such carriers and methods of formulation may be found in Remington The Science and Practice of Pharmacy, 20^{th} edition, Lippincott, Williams & Wilkins, Baltimore Md. To form a pharmaceutically acceptable composition suitable for effective administration, such compositions will contain an effective amount of allopurinol, or other compounds of the disclosure, either with or without modulating compounds.

Pharmaceutical compositions of the invention are administered to a subject in amounts sufficient to treat disorders related to inflammatory conditions in the subject (defined as the “effective amount”). The subject may be a human. In another embodiment, the subject is a mammal. In an alternate embodiment, the subject is an animal. The inflammatory conditions include, but are not limited to, respiratory distress, kidney disease, liver disease, ischemia-reperfusion injury, organ transplantation, sepsis, burns, viral infections, hemorrhagic shock and sickle cell disease. The effective amount may vary according to a variety of factors such as the subject's condition, weight, sex and age. Other factors include the mode or site of administration. The pharmaceutical compositions may be provided to the subject by a variety of routes such as subcutaneous, topical, oral, intraosseous, and intramuscular. Compounds identified according to the methods disclosed herein may be used alone at appropriate dosages defined by routine testing in order to obtain optimal activity, while minimizing any potential toxicity. In addition, co-administration or sequential administration of other agents may be desirable.

The compounds contained in the pharmaceutical compositions discussed herein may be used with or without chemical derivatives. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington The Science and Practice of Pharmacy.

The pharmaceutical compositions containing compounds identified according to this disclosure as the active ingredient for use in the modulation of inflammatory conditions can be administered in a wide variety of therapeutic dosage forms in conventional vehicles for administration. For example, the compounds can be administered in such forms as tablets, capsules (each including timed release and sustained release formulations), pills, powders, granules, elixirs, tinctures, solutions, suspensions, syrups, pastes and emulsions, or by injection internally.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like.

For internal injection, sterile suspensions and solutions are desired. Isotonic preparations that generally contain suitable preservatives are employed when internal injection is desired. The injections may be intravenous or intramuscular.

Topical preparations containing the active drug component can be admixed with a variety of carrier materials well known in the art, such as, e.g., alcohols, aloe vera gel, allantoin, glycerine, vitamin A and E oils, mineral oil, PPG2 myristyl propionate, and the like, to form, e.g., alcoholic solutions, topical cleansers, cleansing creams, skin gels, skin lotions, and shampoos in cream or gel formulations.

Pharmaceutical compositions containing compounds of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. The compounds of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### EXAMPLE 1

The following study has been designed to test the ability of xanthine oxidase inhibitors to restore systemic vascular function in individuals with sickle cell disease by measuring forearm bloodflow rates.

Fifteen sickle cell anemia patients (HbSS, SC, S-β Thalassemia, verified by hemoglobin electrophoresis), >19-years-old, chronic stable patients with hemoglobin>7 gm/dl, non-smokers, normal renal and liver function, not taking hydroxyurea, no blood transfusions in the last 3 months and less than 4 hospitalizations in the past year. Patients will discontinue pain medications 24 hr before study.

Fifteen healthy volunteers (Hb AA, verified by hemoglobin electrophoresis), >19-years-old, normal renal and liver function, normal complete blood count (CBC) with differential, not taking any medications, matched by age, gender, and race. At initial evaluation, both control and SCD subjects will be screened by clinical history, physical examination, CBC with differential, and routine chemical analysis. All subjects will give written informed consent for all procedures.

The forearm blood flow studies are designed to evaluate endothelial function. Similar studies were performed on patients with hypercholesterolemia (249) diabetes (250) and SCD (the latter without allopurinol administration,(251)). All forearm blood flow studies will be performed in the morning in a quiet, temperature-controlled room (˜23° C.). Mornings are selected to avoid the recognized diurnal fluctuation in forearm blood flow (252). Subjects will fast overnight (12 hr, water permitted), refrain from smoking, drinking alcohol or caffeinated beverages for at least 24 hr before the forearm blood flow measurements.

During each forearm blood flow study, infusion of drugs into the brachial artery and measurement of the response of the forearm vasculature by means of strain-gauge venous-occlusion plethysmography will be determined. During each study, infusion of drugs into the brachial artery and measurement of the response of the a) forearm vasculature by means of strain-gauge venous-occlusion plethysmography and b) cerebral blood flow will be determined. All drugs used in this study are approved for human use by the Food and Drug Administration in the form of Investigational New Drugs (IND) and will be prepared by the Pharmaceutical Service of the University of Alabama at Birmingham following procedures to ensure accurate bioavailability and sterility of the solutions.

For forearm blood flow measurements, all subjects will be placed in a supine position and a 20-gauge polytetrafluoroethylene catheter will be cannulated into the brachial artery of the nondominant arm. This arm will be positioned slightly elevated above the level of the right atrium, and a mercury-filled silicone elastomer strain gauge will be placed on the widest part of the forearm. The strain gauge is connected to a plethysmograph (model EC-4, Hokanson) calibrated to measure the percent change in volume and connected in turn to a chart recorder to record flow measurements. For each measurement, an upper arm cuff will be inflated to 40 mmHg with a rapid cuff inflator (model E-10, Hokanson) to occlude venous outflow from the extremity. A pneumatic wrist cuff will be inflated to suprasystolic pressures (200 mm Hg) one minute before each measurement to exclude hand circulation. Flow measurements will be recorded for approximately 7 sec every 15 sec. The mean value of the final five readings will be taken. Baseline measurements will be obtained after a 3 min infusion of 5% dextrose solution at 1 ml/min. Forearm bloodflow will be measured after infusion of sodium nitroprusside (SNP) and acetylcholine (ACh). SNP is an endothelium-independent vasodilator with its effect due to direct action on smooth muscle cells (239). ACh, in contrast, vasodilates by stimulating the release of relaxing factors from the endothelium (240).

SNP will be infused at 0.8, 1.6, and 3.2 μg/mm and ACh chloride (Sigma Chemical Co.) at 7.5, 15, and 30 μg/min (the infusion rates will be 0.25, 0.5, and 1 ml/min, respectively, for each drug). Each dose will be infused for 5 min, and forearm blood flow will be measured during the last 2 min. A 30 min rest period will be allowed and another baseline measurement will be obtained between infusions of the two drugs.

Oxypurinol (Glaxo-Wellcome) dissolved in 5% dextrose will be infused at 300 μg/min (infusion rate, 1 ml/min) for 30 min and baseline flow measurements obtained. This dose is chosen to achieve, at baseline flow conditions, an intravascular concentration of 10 μg/ml. At this concentration, greater than 90% inhibition of xanthine oxidase activity in the forearm blood vessels was found (236), an observation that will be verified in SCD patients for reasons discussed previously. Subsequently, cumulative dose-response curves for ACh and SNP will be repeated during the concomitant infusion of oxypurinol using the same doses, infusion rates, and resting interval. Oxypurinol infusion will be continued during the resting period. The sequence of ACh and SNP infusions, both before and after oxypurinol infusion, will be randomized to avoid bias related to the order of drug infusion. During the study, subjects will be unaware of the drug being infused. All blood pressures will be recorded directly from the intra-arterial catheter after each flow measurement. Forearm vascular resistance will be calculated as the mean arterial pressure divided by the forearm blood flow.

Prior to, during and after the resting period for 30 min, cerebral blood flow will be measured after placing and adjusting the TCD probe. Serial TCD examinations of the intracranial vessels of the circle of Willis will be done to detect right-left asymmetry of flow velocity (FV) in the circle of Willis, the presence of intracranial stenoses, anomalies of the circle of Willis, inadequate collateral circulation, and perfusion from extracranial collateral vessels. This will also allow assessment of the hemodynamic significance of carotid stenosis. The functional status of the intracranial circulation, identification of structural anomalies, and degree of cerebrovascular pathology will be established for each patient. The hand held Doppler exam is performed with the patient recumbent. Doppler spectra will be stored to a hard disk for subsequent analysis. Vital signs and laboratory data will be documented. Arteries examined will include the middle cerebral (MCA) and anterior cerebral (ACA). Transtemporal Doppler windows, thin areas in the skull, will be used for examination of the MCA and ACA during oxypurinol infusion studies. We have observed that in patients with SCD studied that a velocity of >150 cm/sec correlates with significant stenosis. A physician will be present for each examination. Correlation of TCD flows with acute therapy will then be made.

With regard to both phases of this study, a sample size of 15 patients per group was predicted allow detection of an effect a power of ˜80%. This would be associated with a 5% type I error rate. There are approximately 400 adults with SCD in North-Central Alabama, and in the last 12 months, there have been 750 admissions to the University of Alabama Hospital. At the present time, 150 adult SC patients receive regular care at the UAB adult SC clinic, and 75 at Cooper Green Hospital. We anticipate an accrual of at least 20 patients per year; therefore SC patient accrual should be completed within 1 year.

### Materials and Methods

Red Cell Superoxide, Hydrogen Peroxide and Lipid Hydroperoxide Production

Blood was collected from healthy HbA adult volunteers and homozygous HbS patients in anticoagulated (EDTA) vacu-containers. All individuals were evaluated for cytochrome b5 reductatse and glucose-6-phosphate dehydorgenase activity and none were reported deficient. After centrifugation, plasma nad buffy coat were discarded, and cells were washed and filtered through a cellulose column (Sigma, type 50 and a-cellulose) to remove neutrophils and platelets. Packed RBCs were diluted to a hematocrit of 2.5% (vol/vol) hemoglobin concentrations as determined with Drabkin's reagent at 540 nm, and rates of O₂. release over 2 hours were quanitifed spectrophotometrically by CuZn SOD-inhibitable (100 units/ml, equivalent to approximately 33 ug/ml SOD) reduction of cytochrome c (50 uM) at 550 nm(ε_{M}=21 mM⁻¹cm⁻¹). In some experiments, O₂. release was measured in cells pretreated with DMNQ (Oxis, 100 uM), 3-hydroxy-1,2-dimethyl-4-pyridone (HDP, Aldrich, 0.5 mM), and 4,4-diisothiocyano-2,2 disulfonic acid stilbene (DIDS, Sigma, 200 uM). Possible Hb intereference in determination of rates of cytochrome c reduction was evaluated by performing a singular value decomposition analysis (Matlab, Mathworks, Natick, Mass.).

H₂O₂ concentrations were calculated from aminotriazole (AT)-mediated inactivation of catalase activity as described (Royall, et al. Arch. Biochem. Biophys., 294, 686). Red cells were incubated with 10 mM AT at 37 C., and intracellular catalase activity was measured spectrophotometrically based on the consumption of 10 mM H₂O₂ at 240 nm(ε_{M}=43.6 M ⁻¹cm⁻¹).

Additional methods were performed as described in reference 259, which is hereby incorporated by reference.

### REFERENCES

The following references are incorporated by reference herein in their entirety:

1. Freeman B A and Crapo J D: J Biol Chem 256:10986-10992, 1981.

2. Freeman B A and Crapo J D: Lab Invest 47:412-426, 1982.

3. Clancy R M, Leszczynska-Piziak J and Abramson S B: J Clin Invest 90:1116-1121, 1992.

4. Pagano P, Ito Y, Tornheim K, Gallop P, Tauber A and Cohen R: Am J Physiol 268, H2274-2280, 1995.

5. Mohazzab K M, Kaminski P M and Wolin M S: Am J Physiol 266, H2568-H2572, 1995.

6. Schmidt, Hofmann H, Schindler U, Shutenko Z S, Cunningham D D and Feelisch M: Proc Natl Acad Sci USA 93: 14492-14497, 1996.

7. Xia Y, Dawson V L, Dawson T M, Snyder S H and Zweier J L: Proc Nat] Acad Sci USA 93: 6770-6774, 1996.

8. Embury S H, Mohandas N, Paszty C, Cooper P, Cheung A T: J. Clin. Invest. 103:915-920, 1999.

9. Belcher J D, Marker P H, Weber J P, Hebbel R P, Vercellotti G M: Blood. 96:2451-2459, 2000.

10. Hebbel R P, Visser M R, Goodman J L, Jacob H S, Vercellotti G M: J. Clin. Invest. 80:1503-1506.

11. Shiu Y T, Udden M M, McIntire L V: Blood. 95:3232-3241, 2000.

12. Stuart M J, Setty B N: 94:1555-1560, 1999.

13. Platt O S, Brambilla D J, Rosse W F, Milner P F, Castro O, Steinberg M H, Klug P P: N Engl J Med 330:1639-1644, 1994.

14. Kaul D K, Hebbel R P: Clin Invest 106:411-420, 2000.

15. Solovey A, Lin Y, Browne P, Choong S, Wayner E, Hebbel R P: N Engl J Med 337:1584-1590, 1997.

16. Solovey A A, Solovey A N, Harkness J, Hebbel R P: Blood. 97:1937-1941, 2001.

17. Solovey A, Gui L, Key N S, Hebbel R P: J Clin Invest 101:1899-1904, 1998.

18. Johnson C S, Giorgio A J: Arch Intern Med 141:891-893, 1981.

19. Enwonwu C O: Am J Med. Sci 300:366-371, 1990.

20. Hatch F E, Crowe L R, Miles D E, Young J P, Portner M E: Am. J. Hypertens. 2:2-8, 1989.

21. Allon M: Arch. Intern. Med. 150:501-504, 1993.

22. Mantadakis E, Cavender J D, Rogers Z R, Ewalt D H, Buchanan G R: J Pediatr Hematol Oncol 21:518-522, 1999.

23. Rees D C, Cervi P, Grimwade D, O'Driscoll A, Hamilton M, Parker N E, Porter J B: Br J Haematol 91:834-837, 1995.

24. Morris C R, Kuypers F A, Larkin S, Sweeters N, Simon J, Vichinsky E P, Styles L A: Br J Haematol 111:498-500, 2000.

25. Atz A M, Wessel D L: Anesthesiology 87, 988-990, 1997.

26. Glover R E, Ivy E D, Orringer E P, Maeda H, Mason R P: Mol. Pharmacol. 55:1006-1010, 1999.

27. Jiang J, Jordan S J, Barr D P, Gunther M R, Maeda H, Mason R P: Mol Pharmacol 52:1081-1086, 1997.

28. Hebbel R P, Eaton J W, Balasingam M, Steinberg M H: J Clin Invest 70:1253-1259, 1982.

29. Kuross S A, Hebbel R P: Blood 72:1278-1285, 1988.

30. Osarogiagbon U R, Choong S, Belcher J D, Vercellotti G M, Paller M S, Hebbel R P: Blood 1:314-320, 2000.

31. Nath K A, Shah V, Haggard J J, Croatt A J, Smith L A, Hebbel R P, and Katusic Z S: Am J Physiol Regul Integr Com Physiol 279, 81949-81955, 2000.

32. Rubanyi G, Ho E H, Cantor E H, Lumma W C and Botelho L H P: Bichem Biophys Res Commun 181:13921397, 1991.

33. Fujii H, Ichimori K, Hoshiai K and Nakazawa H: J Biol Chem 272:32773-32778, 1997.

34. McCord J M, Fridovich I: J.Biol.Chem. 244:6049-6055, 1969.

35. Granger D N, Rutili G, McCord J M: Gastroenterology 81:2229, 1981.

36. Parks D A, Skinner K A, Tan S, Skinner H B: Xanthine oxidase in biology and medicine, Reactive oxygen species in biological systems. Edited by Gilbert D, Colton C. New York, Kluwer Academic/Plenum, pp 397420, 1999.

37. Yokoyama Y, Beckman J S, Beckman T K, Wheat J K, Cash T G, Freeman B A, Parks D A: Am J Physiol 258:6564-65701990.

38. Sanders S A, Eisenthal R, Harrison R: Eur J Biochem 245:541-548, 1997.

39. Wu X W, Lee C C, Muzny D M, Caskey C T: Proc Natl Acad Sci USA 86:9412-9416, 1989.

40. Tan C M, Xenoyannis S, Feldman R D: Circ Res 77:710 717, 1995.

41. Singh N, Aggarwal S: Int J Cancer 62:107-114, 1995.

42. Wesson D E, Elliott S J: J Pharmacol Exp Ther 270:1197-1207, 1994.

43. Sakai H, Takeguchi N: J Biol Chem 269:23426-23430, 1994.

44. Beedham C: Drug Metabolism Reviews 16:119-156, 1985.

45. Hall W W, Krenitsky T A: Arch.Biochem.Biophys. 251:36-46, 1986.

46. Foppoli C, Coccia R. Cini C, Rosei M A: Biochim Biophys Acta 1334:200-206, 1997.

47. Sakai M, Yamagami K, Kitazawa Y, Takeyama N, Tanaka T: Pharmacol Toxicol 77:36-40, 1995.

48. Wright R M, Vaitaitis G M, Wilson C M, Repine T B, Terada L S, Repine J E: Proc Natl Acad Sci USA 90:10690-10694, 1993.

49. Amaya Y, Yamazaki K, Sato M, Noda K, Nishino T: J Biol Chem. 265:14170-14175, 1990.

50. Terao M, Cazzaniga G, Ghezzi P, Bianchi M, Falciani F, Perani P, Garattini E: Biochem J 283:863-870, 1992.

51. Keith T P, Riley M A, Kreitman M, Lewontin R C, Curtis D, Chambers G: Genetics 116:67-73, 1987.

52. Houde M, Tiveron M C, Bregegere F: Gene 85:391-402, 1989.

53. Sato A, Nishino T, Noda K, Amaya Y: J Biol Chem 270:2818-2826, 1995.

54. Calzi M L, Raviolo C, Ghibaudi E, de Gioia L, Salmona M, Cazzaniga G, Kurosaki M, Terao M, Garattini E: J Bio Chem. 270:3103731045, 1995.

55. Turner N A, Doyle W A, Ventom A M, Bray R C: Eur J Biochem 232:646-657, 1995.

56. Cazzaniga G, Terao M, Lo S P, Galbiati F, Segalla F, Seldin M F, Garattini E: Genomics 23:390-402, 1994.

57. Falciani F, Ghezzi P, Terao M, Cazzaniga G, Garattini E: Biochem J 285:1001-1008, 1992.

58. Dupont G P, Huecksteadt T P, Marshall B C, Ryan U S, Michael J R, Hoidal J R: J Clin Invest. 89:197-202, 1992.

59. Phan S H, Gannon D E, Varam J, Ryan U S, Ward P A: Am J Pathol 134:1201-1211, 1989.

60. Jarasch E D, Bruder G, Held H W: Acta Physiol Scand Supplementum 548:39-46, 1986.

61. Ghezzi P, Bianchi M, Mantovani A, Spreafico F, Salmona M: Biochem Biophys Res Commun 119:144-149, 1984.

62. Parks D A, Granger D N: Xanthine oxidase: Acta Physiol Scand Supplementum 548:87-99, 1986.

63. Delona L, Abbott V, Gooderham N, Mannering G J: Biochem Biophys Res Commun. 131:109-114, 1985.

64. Ghezzi P, Dinarello C A, Bianchi M, Rosandich M E, Repine J E, White C W: Cytokine 3:189-194, 1991.

65. Moochhala S, Renton K W: Int J Immunopharmacol 13:903-912, 1991.

66. Palmer L A, Johns R A: Chest 114:33S-345, 1998.

67. Jarasch E D, Grund C, Bruder G, Held H W, Keenan T W, Franke W W: Cell 25:67-82, 1981.

68. Bruder G, Held H W, Jarasch E D, Mather I H: Differentiation 23:218-225, 1983.

69. Clare D A, Lecce J G: Arch Biochem Biophys 286:233-237, 1991.

70. Zimmerman B J, Parks D A, Grisham M B, Granger D N: Am J Physiol 255:H202-H2061988.

71. Grum C M, Ragsdale R A, Ketai L H, Shlafer M: Biochem Biophys Res Commun 141:1104-1108, 1986.

72. Emerit I, Fabiani J N, Ponzio O, Murday A, Lunel F, Carpentier A: Ann Thorac Surg 46:619-624, 1988.

73. Eddy L J, Stewart J R, Jones H P, Engerson T D, McCord J M, Downey J M: Am J Physiol 253:H709-H7111987.

74. Rouquette M, Page S, Bryant R, Benboubetra M, Stevens C R, Blake D R, Whish W D, Harrison R, Tosh D: FEBS Lett 426:397-401, 1998.

75. Partridge C A, Blumenstock F A, Malik A B: Arch Biochem Biophys 294:184-187, 1992.

76. Giler S, Sperling O, Brosh S, Urca I, De Vries A: Clin Chim Acta 63:3740, 1975.

77. Ramboer C, Piessens F, De Groote J: Digestion 7:183-195, 1972.

78. Tan S, Gelman S, Wheat J K, Parks D A: South Med J 88:479-482, 1995.

79. Grum, C. M., Ragsdale, R. A., Ketai, L. H., and Simon, R. H: J Critical Care 2:22-26, 1987.

80. Friedl H P, Smith D J, Till G O, Thomson P D, Louis D S, Ward P A: Am J Pathol 136:491-495, 1990.

81. Terada L S, Dormish J J, Shanley P F, Leff J A, Anderson B O, Repine J E: Am J Physiol 263:L394-L401, 1992.

82. Akaike T, Ando M, Oda T, Doi T, Ijiri S, Araki S, Maeda H: J Clin Invest 85:739-745, 1990.

83. Anderson B O, Moore E E, Moore F A, Leff J A, Terada L S, Harken A H, Repine J E: J Appl Physiol 71:18621865, 1991.

84. Tan S, Yokoyama Y, Dickens E, Cash T G, Freeman B A, Parks D A: Free Radic Biol Med 15:407-414, 1993.

85. Cunha F Q, Moss D W, Leal L M, Moncada S, Liew F Y: Immunology 78:563-567, 1993.

86. Jialal I, Freeman D A, Grundy S M: Arterioscler Thromb 11:482-488, 1991.

87. Nielsen V G, Weinbroum A, Tan S, Samuelson P N, Gelman S, Parks D A: J Thorac Cardiovasc Surg 107:1222-1227, 1994.

88. Nielsen V G, McCammon A T, Tan S, Kirk K A, Samuelson P N, Parks D A: J Thorac Cardiovasc Surg 110:715-722, 1995.

89. de Groot H, Littauer A: Biochem Biophys Res Commun 155:278-282, 1988.

90. Fridovich I: Arch Biochem Biophys 247:1-11, 1986.

91. Quinn M T, Parthasarathy S, Fong L G, Steinberg D: Proc Natl Acad Sci USA 84:2995-2998, 1987.

92. Hook M, Kjellen L, Johansson S: Annu Rev Biochem 53:847-869, 1984.

93. Saxena U, Klein M G, Goldberg I J: J Biol Chem 265:12880-12886, 1990.

94. Stern D M, Drillings M, Nossel H L, Hurlet-Jensen A, LaGamma K S, Owen: Proc Natl Acad Sci USA 80:4119-4123, 1983.

95. Ryan J, Wolitzky B, Heimer E, Lambrose T, Felix A, Tam J P, Huang L H, Nawroth P, Wilner G, Kisiel W: J Biol Chem 264:20283-20287, 1989.

96. Schwalbe R A, Ryan J, Stern D M, Kisiel W, Dahlback B, Nelsestuen G L: J Biol Chem 264:20288-20296, 1989.

97. Schnitzer J E, Carley W W, Palade G E: Am J Physiol 254:H425-H437, 1988.

98. Karlsson K, Marklund S L: Lab Invest 60:659-666, 1989.

99. Adachi T, Marklund S L: J Biol Chem 264:8537-8541, 1989.

100. Weinbroum A, Nielsen V G, Tan S, Gelman S, Matalon S, Skinner K A, Bradley E J, Parks D A: Am J Physiol 268:6988-6996, 1995.

101. Nielsen V G, Tan S, Baird M S, Samuelson P N, McCammon A T, Parks D A: Crit Care Med 25:1044-1050, 1997.

102. Ichimori K, Fukahori M, Nakazawa H, Okamoto K, Nishino T: J Biol Chem 274:7763-7768, 1999.

103. Cote C G, Yu F-S, Zulueta J J, Vosatka R J, Hassoun P M: Am J Physiol 271:L869-L874, 1996.

104. Houston M, Chumley P, Radi R, Rubbo H, Freeman B A: Arch Biochem Biophysiol 355:1-8, 1998.

105. Trujillo M, Alvarez M N, Gonzalo P, Freeman B A, Radi R: J Biol Chem 273:7828-7834, 1998.

106. Godber B L J, Doel J J, Sapkota G P, Blake D R, Stevens C R, Eisenthal R, Harrison R: J Biol Chem 275:7757-7763, 2000.

107. Millar T M, Stevens C R, Benjamin N, Eisenthal R, Harrison R, Blake D R: FEBS Letters 427:225-228, 1998.

108. Cannon R O III. Schechter A N, Panza J A, Ognibene F P, Pease-Fye M E, Waclawiw M A, Shelhamer J H, Gladwin M T: J Clin Invest 108:279-287, 2001.

109. Kelley A N: Nephron 14:99-115, 1975.

110. Mize C, Johnson J L, Rajagopalan K V: J Inherit Metab Dis 18:283-290, 1995.

111. van der Vliet A, Smith D, O'Neill C A, Kaur H, Darley-Usmar V, Cross C E, Halliwell B: Biochem J 303:295-301, 1994.

112. Kaur H, Halliwell B: Chem Biol Interact. 73:235-247, 1990.

113. Becker B F, Reinholz N, Leipert B, Raschke P, Permanetter B, Gerlach: Chest 100:176S-181 S, 1991.

114. Hicks M, Wong L S, Day R O: Free Radic Res Commun 18:337-351, 1993.

115. Verdecchia P, Schillaci G, Reboldi G, Santeusanio F, Porcellati C, Brunetti P: Hypertension. 36:10721078, 1000.

116. Aldermnan M H, Cohen H, Madhavan S, Kivlighn S: Hypertension. 34:144-150, 1999.

117. Johnson R J, Kivlighn S D, Kim Y G, Suga S, Fogo A B: Am J Kid Dis 33:225-234, 1999.

118. Lehto S, Niskanen L, Ronnemaa T, Laakso M: Stroke 29:635-639, 1998.

119. Doehner W, Rauchhaus M, Florea V G, Sharma R, Bolger A P, Davos C H, Coats A J S, Anker S D: Am Heart J 141:792-799, 2001.

120. Reynolds M D: Semin Arthritis Rheum 12:404413, 1983.

121. Diamond H S, Meisel A D, Holden D: Ann Intern Med 90:752-757, 1979.

122. Diamond H S, Meisel A, Sharon E, Holden D, Cacatian A: Am J Med 59:796-802, 1975.

123. Malech H L, and Gallin J I: New Engl J Med 317: 687-694, 1987.

124. Sibille Y and Reynolds H Y: Amer Rev Resp Dis 141:471-501, 1990.

125. Strieter R M, Lukacs N W, Standiford T J, and Kunkel S L: Thorax 48:765-769, 1993.

126. Hogg J C: Am J Radio] 163:769-775, 1994.

127. Barnes P J: Free Rad Biol Med 9: 235-243, 1990.

128. Miller R A and Britigan B E: J. Invest Med 43: 39-49, 1995.

129. Babior B M: NADPH Oxidase: Blood. 93:1464-1476. 1999

130. Weiss S J, Test S T, Eckmann C M, Roos D, and Regiani S: Science 234: 200-203, 1986.

131. Weiss S J, Lampert M B, and Test S T: Science 222:625-628, 1983.

132. Cooke J P, Dzau V J: Annu Rev Med 48:489-509, 1997.

133. Stuehr D J: Biochim.Biophys.Acta 1411:217-230, 1999.

134. Gaston B, Reilly J, Drazen J M, Fackler J, Ramdev P, Arnelle D, Mullins M E, Sugarbaker D J, Chee C, Singel D J, et al.: Proc Natl Acad SciUSA 90:10957-10961, 1993.

135. Marley R, Feelisch M, Holt S, Moore K: Free Radic Res 32:1-9, 2000.

136. Singh K, Balligand J L, Fischer T A, Smith T W, Kelly R A: J Biol Chem 271:1111-1117, 1996.

137. Miles A M, Owens M W, Milligan S, Johnson G G, Fields J Z, Ing T S, Kottapalli V, Keshavarzian A, Grisham M B: J Leuk Biol 58:616622, 1995.

138. Carreras M C, Pargament G A, Catz S D, Poderoso J J, Boveris A: FEBS Lett. 341:65-68, 1994.

139. Goode H F, Webster N R, Howdle P D, Walker B E: Clin Sci 86:411-415, 1994.

140. Wright C D, Mulsch A, Busse R, Osswald H: Biochem Biophys Res Commun 160:813-819, 1989.

141. Bryant, J L, Mehta P, Von der Porten A, and Mehta J L: Biochem Biophys Res Commun 189:558-64, 1992.

142. Schmidt H H H W, Seifert R, and Bohme E: FEB 244:357-360, 1989.

143. Stuehr D J and Marietta M A. Mammalian nitrate biosynthesis: Proc Natl Acad Sci USA 82: 7738-7742, 1985.

144. Munoz-Fernandez M A, Fernandez M A and Fresno M: Eur J Immunol 22:301-307, 1992.

145. Tayeh M A and Marietta M A: J Biol Chem 264(33):19654-19658, 1989.

146. Nathan C: FASEB Journal 6:3051-3064, 1992.

147. Nussler A K, Billiar T R: J Leuk Biol 54:171-178, 1993.

148. Patel R P, McAndrew J, Sellak H, White C R, Jo H, Freeman B A, Darley-Usmar V M: Biochim Biophys Acta 1411:385-400, 1999.

149. Kennedy M C, W E Antholine and Helmut Beinert: J Biol Chem 272:20340-20347, 1997.

150. Castro L, Rodriguez M, and Radi R: J Biol Chem 269: 29409-29415, 1994.

151. Hausladen A and Fridovich I: J Biol Chem 269:29405-29408, 1994.

152. Beckman J S, Beckman T W, Chen J, Marshall P A and Freeman B A. Proc Natl Acad Sci 87:1620-1624, 1990.

153. Radi R, Beckman J S, Bush K M and Freeman B A. J Biol Chem 266:4244-4250, 1991.

154. Radi R, Beckman J S, Bush K M and Freeman B A. Arch Biochem Biophys 288:481-487, 1991.

155. Eiserich J P, Hristova M, Cross C E, Jones A D, Freeman B A, Halliwell B and van der Vliet A. Nature 391, 393-397, 1998.

156. Beckman J S, Ischiropoulos H, Zhu L, van der Woerd M, Smith C D, Chen J D, Harrison J, Martin J C, and Tsai. M: Arch Biochem Biophys 298: 438-445, 1992.

157. Ischiropoulos H: Arch Biochem Biophys. 356:1-11, 1998.

158. Patel R K, McAndrew J, Sellak H, White C R, Jo H, Freeman B A, Darley-Usmar V: Biochem Biophys Acta. 447:1-16, 1999.

159. Rubbo H, Darley-Usmar V and Freeman B A. Chem Res Toxicol 9:809-820, 1996.

160. van der Vliet A, Eiserich J P, Shigenaga M K and Cross C E: Am J Respir Crit Care Med 159:1-9, 1999.

161. Koppenol W H, Moreno J J, Pryor W A, Ischiropoulos H and Beckman J S: Chem Res Toxicol 5: 834-842, 1992.

162. Kissner R, Nauser T, Bugnon P, Lyle P G, and Koppenol W H: Chem Res Toxicol 10:1285-1292, 1997.

163. Rubbo H, Parthasarathy S, Kalyanaraman B, Barnes S, Kirk M and Freeman B A: Arch Biochem Biophys 324: 15-25, 1995.

164. Rubbo H, Radi R, Trujillo M, Telleri R, Kalyanaraman B, Barnes S, Kirk M and Freeman B A: J Biol Chem 269: 26066-26075, 1994.

165. Stamler J S: Cur Top Mic Immunol 196: 19-36, 1995.

166. Beckman J S, Ischiropoulos H, Zhu L, van der Woerd M, Smith C D, Chen J D, Harrison J, Martin J C and Tsai M. Arch Biochem Biophys 298: 438-445, 1992.

167. Beckman J S, Ye Y, Anderson P G, Chen J, Accavitti M A, Tarpey M M and White R. Biol Chem Hoppe-Seyler 375: 81-88, 1994.

168. Alvarez B, Rubbo H, Kirk M, Barnes S, Freeman B A and Radi R: Chem Res Toxicol 9:390-396, 1996.

169. Shigenaga M K, Lee H, Blount B, Christen S, Shigeno E T, Yip H and Ames B N: Proc Natl Acad Sci USA (In Press).

170. Leeuwenburgh C, Hardy M M, Hazen S L, Wagner P, Oh-ishi S, Steinbrecher U P and Heinecke J W: J Biol Chem 272:1433-1436, 1997.

171. Christen S, Woodall A A, Shigenaga M K, Southwell-Keely P T, Duncan M W and Ames B N: Proc Natl Acad Sci USA 94:3217-3222, 1997.

172. Rubanyi G M, Ho E H, Cantor E H, Lumma W C, Botelho L H: Biochem Biophys Res Commun 181:13921397, 1991.

173. Fujii H, Ichimori K, Hoshiai K, Nakazawa H: J Biol Chem 272:32773-32778, 1997.

174. Albina J E, Reichner J S: New Horizons 3:46-64, 1995.

175. Flavahan N A: Circulation 85:1927-1938, 1992.

176. Cooke J P, Singer A H, Tsao P, Zera P, Rowan R A, Billingham M E: J Clin Invest 90:1168-1172, 1992.

177. Kubes P, Suzuki M, Granger D N: Proc Natl Acad Sci USA 88:4651-4655, 1991.

178. Siegfried M R, Carey C, Ma X L, Lefer A M: Am J Physiol 263:H771-H777, 1992.

179. Lefer D J, Nakanishi K, Vinten-Johansen J: J Cardiovasc Pharmacol 22 Suppl 7:S34-S43, 1993.

180. Kurose I, Wolf R, Grisham M B, Granger D N: Circ Res 74:376-382, 1994.

181. Niu X F, Ibbotson G, Kubes P: Circ Res 79:992-999, 1996.

182. Yacoub M, Edelstein C L, Schrier R W: Nephrol Dial Transplant 11:1738-1742, 1996.

183. Radi R, Beckman J S, Bush K M, Freeman B A: Arch Biochem Biophys 288:481 487, 1991.

184. Rubbo H, Parthasarathy S, Barnes S, Kirk M, Kalyanaraman B, Freeman B A: Arch Biochem Biophys 324:15-25, 1995.

185. Rubbo H, Radi R, Trujillo M, Telleri R, Kalyanaraman B, Barnes S, Kirk M, Freeman B A: J Biol Chem 269:26066-26075, 1994.

186. Gergel D, Misik V, Ondrias K, Cederbaum A I: J Biol Chem 270:20922-20929, 1995.

187. Darley-Usmar V M, Patel R P, O'Donnell V B, and Freeman B A. Antioxidant actions of nitric oxide. In: Nitric Oxide. Ed. L J Ignarro, Academic Press, 2000, pp 265-276.

188. Quyyumi, A. A., N. Dakak: J Clin Invest 95(4): 1747-55.

189. Panza, J. A., P. R. Casino: Circulation 87(5): 1468-74.

190. Britten, M. B., A. M. Zeiher, et al.: J Intern Med 245(4): 315-27.

191. Schachinger, V., M. B. Britten, et al.: Circulation 100(14): 1502-8.

192. Schachinger, V., M. B. Britten, et al.: Circulation 10 1(16): 1899-906.

193. Enwonwu, C. O., X. X. Xu, et al.: Am J Med Sci 300(6): 366-71.

194. Lopez, B. L., J. Barnett, et al.: Acad Emerg Med 3(12): 1098-103.

195. Lopez, B. L., L. Davis-Moon, et al.: Am J Hematol 64(1): 15-9.

196. Morris, C. R., F. A. Kuypers, et al.: Br J Haematol 1 11(2): 498-500.

197. Morris, C. R., F. A. Kuypers, et al.: J Pediatr Hematol Oncol 22(6): 515-20.

198. Bank, N., H. S. Aynedjian, et al.: Kidney Int 50(1): 184-9.

199. Nagel, R. L.: J Clin Invest 104(7): 847-8.

200. Kaul, D. K. and R. P. Hebbel: J Clin Invest 106(3): 411-420.

201. Ryan, T. M., D. J. Ciavatta, et al.: Science 278(5339): 873-6.

202. De Caterina, R., P. Libby, et al. ; J Clin Invest 96(1): 60-8.

203. Stuart, M. J. and B. N. Setty: Blood 94(5): 1555-60 204.

204. Space, S. L., P. A. Lane, et al.: Am J Hematol 63(4): 200-4

205. French, J. A., 2nd, D. Kenny, et al.: Blood 89(12): 4591-9.

206. Holzman, S. L., H. Shen, et al. (2000). Blood 96(11): 528a.

207. Baldus S, Castro L, Eiserich J P and Freeman B A: Am J Respir Crit Care Med, 163:308-310, 2001.

208. Eiserich J P, Estevez A G, Bamberg T V, Ye Y Z, Chumley P H, Beckman J S and Freeman B A: Proc Natl Acad Sci USA. 96:6385-6370, 1999.

209. Sampson, J B, Rosen H and Beckman J S: Meth Enzymol 269:210-218, 1996.

210. Radi R, Cosgrove T P, Beckman J S and Freeman B A: Biochem J 290: 51-57, 1993.

211. Denicola A, Freeman B A, Trujillo M, and Radi R. Arch Biochem Biophys 333: 49-58, 1996.

212. Alvarez, B, Ferrer-Sueta G, Freeman B A and Radi R: J Biol Chem 274:842-848, 1999.

213. Lang J D Jr., Chumley P, Eiserich J P, Estevez A, Bamberg T, Adham A, Crow J and Freeman B A. Am J Physiol: Lung Cell Mol Physiol 279:L994-L1002, 2000.

214. Baldus S, Eiserich J P, Castro L, Jackson R M, Figueroa M, Chumley P, Ma W, Tousson A, White C R, Freeman B A: Submitted, J Clin Invest, 2001.

215. Eiserich J P, Baldus S, Brennan M-L, Ma W, Zhang C, Tousson A, Castro L, Lusis A J, White C R, and Freeman B A: Submitted, Science, 2001.

216. Eiserich J P, Hristova M, Cross C E, Jones A D, Freeman B A, Halliwell B and van der Vliet A: Nature 391:393-397, 1998.

217. van der Vliet A, Eiserich J P, Halliwell B and Cross C E: J Biol Chem 272: 7617-7625, 1997.

218. Sampson J B, Ye Y Z, Rosen H and Beckman J S: Arch Biochem Biophys 356: 207-213, 1998.

219. Bloodsworth A, O'Donnell V and Freeman B A. Arterioscler Thromb Vase Biol 20:1707-1715, 2000.

220. O'Donnell V B and Freeman B A. Circ Res 88:12-21, 2001.

221. O'Donnell V B; Coles B, Lewis M J, Crews B C, Mamett L J and Freeman B A: J Biol Chem 275:38239-38244, 2000.

222. O'Donnell V B, Taylor K B, Parthasarathy S, Kuhn H, Koesling D, Friebe A., Bloodsworth A, Darley-Usmar V M and Freeman B A: J Biol Chem 274:20083-20091, 1999.

223. Coffey M J, Chumley P H, Coles B, Natarajan R, Thimmalapura P, Nowell M, Kohn H, Lewis M J, Freeman B A, O'Donnell V B: Proc Nat Acad Sci 98:8006-8011, 2001.

224. Nielsen V G, Tan S, Weinbroum A, McCammon A T, Samuelson P N, Gelman S, Parks D A: Am J Respir Crit Care Med 154:1364-1369, 1996.

225. Pesonen E J, Linder N, Raivio K O, Samesto A, Lapatto R, Hockerstedt K, Makisalo H, Andersson S: Gastroenterology 114:1009-1015, 1998.

226. White C R, Darley-Usmar V, Berrington W R, McAdams M, Gore J Z, Thompson J A, Parks D A, Tarpey M M, Freeman B A: Proc.Natl.Acad.Sci.USA 93:8745-8749, 1996.

227. Sheng K, Shariff M, Hebbel R P: Blood 91:3467-3470, 1998.

228. Radi R, Rubbo H, Bush K, Freeman B A: Arch Biochem Biophys 339:125-135, 1997.

229. Houston M, Estevez A, Chumley P, Aslan M, Marklund S, Parks D A, Freeman B A: J Biol Chem.274:4985-4994, 1999.

230. Tarpey M M, Beckman J S, Ischiropoulos H, Gore J Z, Block T A: FEBS Lett. 364:314-318, 1995.

231. Suh Y A, Arnold R S, Lassegue B, Shi J, Xu X, Sorescu D, Chung A B, Griendling K K, Lambeth J D: Nature 401:79-82, 1999.

232. Banfi B, Maturana A, Jaconi S, Amaudeau S, Laforge T, Sinha B, Ligeti E, Demaurex N, Krause K H: Science 287:138-142, 2000.

233. Mohazzab K M, Kaminski P M, Wolin M S: Am J Physiol 266:H2568-H25721994.

234. Munzel T, Hink U, Heitzer T, Meinertz T: Ann NY Acad Sci 874:386-400, 1999.

235. Panus P C, Wright S A, Chumley P H, Radi R, Freeman B A: Arch Biochem Biophys 294:695-702, 1992.

236. Frost M T, Halliwell B, Moore K P. Biochem J 345:453-458, 2000.

237. Beckman J S, Parks D A, Pearson J, Marshall P and Freeman B A: Free Radic Biol Med 6:607-615, 1989.

238. Zollner O, Lenter M C, Blanks J E, Borges E, Steegmaier M, Zer-wes H G, Vestweber D: J Cell Biol 136:707-716, 1997.

239. Ding Z M, Babensee J E, Simon S I, Lu H, Perrard J L, Bullard D C, Dal X Y, Bromley S K, Dustin M L, Entman M L, Smith C W, Ballantyne C M: J Immunol 163:5029-5038, 1999.

240. Kalogeris T. J, Kevil C G, Laroux F S, Coe L L, Phifer T J, Alexander J S: Am J Physiol 276:L9-L19, 1999.

241. Luscinskas F W, Kansas G S, Ding H, Pizcueta P, Schleiffenbaum B E, Tedder T F, Gimbrone M A Jr: J Cell Biol 125:1417-1427, 1994.

242. Kevil C G, Okayama N, Trocha S D, Kalogeris T J, Coe L L, Specian R D, Davis C P, Alexander J S: Microcirculation 5:197-210, 1998.

243. Kevil C G, Payne D K, Mire E, Alexander J S: J Biol Chem] 273:15099-15103, 1998.

244. Royal] J A, R L Berkow, J S Beckman, M K Cunningham, S Matalon and B A Freeman: Am J Physiol Lung Cell Mol Biol 257:L399-L410, 1989.

245. Eppihimer M J, Wolitzky B, Anderson D C, Labow M A, Granger D N: Circ Res 79:560-569, 1996.

246. Henninger D D, Panes J, Eppihimer M, Russell J, Gerritsen M, Anderson D C, Granger D N: J Immunol 158:1825-1832, 1997.

247. Tarpey M M, Fridovich I: Circ Res 89:224-236, 2001.

248. White C R, Brock T A, Chang L-Y, Crapo J, Briscoe P, Ku D, Bradley W A, Gianturco S H, Gore J, Freeman B A, Tarpey M M: Proc Natl Acad Sci 91:1044-1048, 1994.

249. Cardillo C, Kilcoyne C M, Cannon R O, Quyyumi A A, Panza J A: Hypertension 30:57-63, 1997.

250. Butler R, Morris A D, Belch J J F, Hill A, Struthers A D: Hypertension 35:746-751, 2000.

251. Belhassen L, Pelle G, Sediame S, Bachir D, Carville C, Bucherer C, Lacombe C, Galacteros F, Adnot S: Blood 97:1584-1589, 2001.

252. Mohan J S, Marshall J M, Reid H L, Serjeant G R: Clin Auton Res 5:129-134, 1995.

253. Bohme E, GrafH, Schultz G: Adv Cyclic Nucl Res 9:131-143, 1978.

254. Furchgott R F, Zawadzki J V: Nature 288:373-376, 1980.

255. Day R O, Miners J, Birkett D J, Graham G G, Whitehead A: Br J Pharmacol 26:429434, 1988.

256. Rundles R W. Arch Intern Med. 145(8):1492-503, 1985.

257. Lopez B L, Barnett J, Ballas S K, Christopher T A, Davis-Moon, Ma X: Acad Emerg Med. 3(12):1098-103, 1996.

258. Lopez B L, Davis-Moon L, Ballas S K, Ma X L: Am J Hematol. 64(1):15-19, 2000.

259. Aslan M, Ryan T M, Adler B, Townes T M, Parks D A, Thompson J A, Tousson A, Gladwin M T, Patel R P, Tarpey M M, Batinic-Haberle, White C R, Freeman B A: Proc Natl Acad Sci USA, 98(26) 15215-15220 (2001).

260. Arellano F, Sacristan J A: Ann Pharmacother. 27(3):337-43, 1993.

261. Wolkenstein P, Revuz J: Drug Saf. 13(1):56-68, 1995.

What is claimed:

1. A method of treating an inflammatory condition in a subject in need of such treatment comprising administering to the subject an effective amount of allopurinol, or a pharmaceutically acceptable salt thereof, under conditions such that said treatment is effected.

2. The method according to claim 1 wherein said inflammatory condition is sickle cell disease.

3. The method of claim 2 where the sickle cell disease involves a xanthine oxidase and allopurinol inhibits the activity of xanthine oxidase.

4. The method of claim 1 where the inflammatory condition is selected from the group consisting of respiratory distress, kidney disease, liver disease, ischemia-reperfusion injury, organ transplant, sepsis, burns, viral infections and hemorrhagic shock.

5. The method of claim 4 where the inflammatory condition involves xanthine oxidase and allopurinol inhibits xanthine oxidase activity.

6. The method of claim 1 where the subject is human.

7. The method of claim 1 where the subject is a mammal.

8. A pharmaceutical composition for the treatment of sickle cell disease comprising allopurinol, or a pharmaceutically acceptable salt thereof, a pharmaceutically acceptable carrier thereof.

9. The pharmaceutical composition of claim 8 further comprising a modulating compound.

10. The pharmaceutical composition of claim 9 where the modulating compound is oxypurinol, or a pharmacologically acceptable salt thereof.

11. A pharmaceutical composition for the treatment of sickle cell disease comprising oxypurinol, or a pharmaceutically acceptable salt thereof, and a pharmaceutically acceptable carrier thereof.

12. The pharmaceutical composition of claim 11 further comprising a modulating compound.

13. The pharmaceutical composition of claim 12 where the modulating compound is allopurinol, or a pharmacologically acceptable salt thereof.

14. A method of maintaining a biological function of .NO, or active forms thereof, in a subject comprising administering to the subject an effective amount of allopurinol, or a pharmaceutically acceptable salt thereof, to effect said maintenance.

15. The method of claim 14 where said maintenance involves inhibiting a xanthine oxidase activity.

16. The method of claim 15 where the xanthine oxidase activity is increased by an inflammatory condition selected from the group consisting of sickle cell disease, respiratory distress, kidney disease, liver disease, ischemia-reperfusion injury, organ transplant, sepsis, bums, viral infections and hemorrhagic shock.

17. The method of claim 14 where the subject is human.

18. The method of claim 14 where the subject is a mammal.

19. A method of protecting a subject from oxidative stress comprising administering to the subject an effective amount of allopurinol, or a pharmaceutically acceptable salt thereof, to effect said protection.

20. The method of claim 19 where said protection involves inhibiting a xanthine oxidase activity.

21. The method of claim 20 where the xanthine oxidase activity is increased by an inflammatory condition selected from the group consisting of sickle cell disease, respiratory distress, kidney disease, liver disease, ischemia-reperfusion injury, organ transplant, sepsis, burns, viral infections and hemorrhagic shock.

22. The method of claim 19 where the subject is human.

23. The method of claim 19 where the subject is a mammal.

24. A method of restoring vascular function in a subject suffering from sickle cell disease comprising administering to the subject an effective amount of allopurinol, or a pharmaceutically acceptable salt thereof, to effect said restoration.

25. The method of claim 24 where said restoration involves inhibiting a xanthine oxidase activity.

26. The method of claim 24 where the subject is human.

27. The method of claim 24 where the subject is a mammal.
